(19) United States
(12) Patent Application Publication
(10) Pub. No.: US 2026/0102177 A1
(43) Pub. Date: Apr. 16, 2026

(54) END TOOL FOR SURGICAL INSTRUMENT

(71) Applicant: LIVSMED INC., Seongnam-si (KR)

(72) Inventors: Tae Jin PARK, Seongnam-si (KR); Hee Jin KIM, Seoul (KR); Dong Hoon KANG, Seoul (KR); Jae Yeong LEE, Seongnam-si (KR)

(21) Appl. No.: 19/357,874

(22) Filed: Oct. 14, 2025

(30) Foreign Application Priority Data
Oct. 14, 2024 (KR) 10-2024-0139781

(51) Int. Cl. A61B 17/29; A61B 17/00

(52) U.S. Cl. CPC A61B 17/29; A61B 2017/00367; A61B 2017/00477; A61B 2017/2929; A61B 2017/2933; A61B 2017/2944

(57) ABSTRACT

An end tool for a surgical instrument includes an end tool hub having a first jaw coupling pulley, a second jaw coupling pulley, a first jaw auxiliary pulley assembly, and a second jaw auxiliary pulley assembly rotatably positioned thereon, the end tool hub being configured to guide a first path of a first jaw first wire portion and a second path of a first jaw second wire portion, both extending from the first jaw auxiliary pulley assembly, to one side of a first reference plane defined as an imaginary plane, and to guide third path of a second jaw first wire portion and a fourth path of a second jaw second wire portion, both extending from the second jaw auxiliary pulley assembly, to another side of the first reference plane.

### CROSS-REFERENCE TO RELATED APPLICATION

This application is based on and claims priority under 35 USC §119 to Korean Patent Application No. 10-2024-0139781, filed on October 14, 2024, in the Korean Intellectual Property Office, the disclosure of which is incorporated by reference herein in its entirety.

### BACKGROUND

### Technical Field

The present disclosure relates to an end tool for a surgical instrument, and more particularly, to an end tool for a surgical instrument that is mountable on a robot arm or operable manually, for use in laparoscopic surgery or various other surgical procedures.

### Background Art

Medically, surgery refers to the process of cutting, slitting, or manipulating the skin, mucous membranes, or other tissues using medical devices to treat diseases. In particular, open surgeries, which involve incising the skin at a surgical site to access and treat, reconstruct, or remove an internal organs, may cause bleeding, side effects, patient pain, scars, or other issues. Accordingly, minimally invasive surgeries performed by creating a small hole in the skin and inserting a medical device, such as a laparoscopic surgical instrument, a microsurgical microscope and the like, or surgeries performed using a robot have recently been spotlighted as alternatives.

A surgical instrument is a tool equipped with an end tool provided on one end of a shaft that passes through a hole drilled in the skin, and manipulated by a surgeon by hand using a predetermined driving part or by a robot arm to perform surgeries at the surgical site. The end tool provided on the surgical instrument performs a rotational motion, a gripping motion, a cutting motion, or the like through a predetermined structure.

However, in conventional surgical instruments, end tool portions are not bendable, which makes it difficult to access the surgical site and perform various surgical operations. To address this issue, surgical instruments with bendable end tool portions have been developed. However, the operation of a manipulation part for bending an end tool or performing surgical motions does not intuitively correspond to the actual bending of the end tool or the execution of surgical motions, making it difficult for a surgeon to operate intuitively and requiring a long period of training to become proficient.

The background art described above is technical information retained by the present inventors in order to derive the present disclosure or obtained by the present inventors in the process of deriving the present disclosure, and thus is not necessarily known art disclosed to the general public before the filing of the present disclosure.

### SUMMARY

The present disclosure is directed to providing an end tool for a surgical instrument that is mountable on a robot arm or operable manually for use in laparoscopic or various other surgical procedures, the end tool capable of increasing a rotation radius of jaws by adjusting the arrangement of jaw wire paths.

According to an aspect of the present disclosure, an end tool for a surgical instrument may include a first jaw and a second jaw that are rotatable independently of each other, a first jaw coupling pulley which is coupled to the first jaw and to which a first jaw wire having a first jaw first wire portion and a first jaw second wire portion is coupled, a second jaw coupling pulley which is coupled to the second jaw and to which a second jaw wire having a second jaw first wire portion and a second jaw second wire portion is coupled, a first jaw auxiliary pulley unit on which the first jaw wire extending from the first jaw coupling pulley is wound and configured to adjust a path of the first jaw first wire portion and a path of the first jaw second wire portion, a second jaw auxiliary pulley unit on which the second jaw wire extending from the second jaw coupling pulley is wound and configured to adjust a path of the second jaw first wire portion and a path of second jaw second wire portion, and an end tool hub in which the first jaw coupling pulley, the second jaw coupling pulley, the first jaw auxiliary pulley unit, and the second jaw auxiliary pulley unit are rotatably positioned, the end tool hub being configured to guide the paths of the first jaw first wire portion and the first jaw second wire portion, which extend from the first jaw auxiliary pulley unit, to one side of a first reference plane, which is defined as an imaginary plane, and to guide the paths of the second jaw first wire portion and the second jaw second wire portion, which extend from the second jaw auxiliary pulley unit, to another side of the first reference plane.

In another embodiment of the present disclosure, the first jaw auxiliary pulley unit (or assembly) may include a first jaw first auxiliary pulley configured to guide the first jaw first wire portion, which extends from the another side of the first reference plane while being wound around the first jaw coupling pulley, to the one side of the first reference plane, and to guide the first jaw second wire portion, which extends from the one side of the first reference plane while being wound around the first jaw coupling pulley, to the another side of the first reference plane, and the second jaw auxiliary pulley unit (or assembly) may include a second jaw first auxiliary pulley configured to guide the second jaw first wire portion, which extends from the another side of the first reference plane while being wound around the second jaw coupling pulley, to the one side of the first reference plane, and to guide the second jaw second wire portion, which extends from the one side of the first reference plane while being wound around the second jaw coupling pulley, to the another side of the first reference plane.

In the other embodiment of the present disclosure, the end tool hub may include a first main guide groove defined on the one side of the first reference plane and configured to guide the path of the first jaw second wire portion extending from the first jaw first auxiliary pulley, and a second main guide groove defined on the another side of the first reference plane and configured to guide the path of the second jaw first wire portion extending from the second jaw first auxiliary pulley.

In the other embodiment of the present disclosure, the first jaw auxiliary pulley unit (or assembly) may further include a first jaw guide pulley rotatably coupled to the end tool hub and configured to guide the path of the first jaw second wire portion, which extends from the first jaw first auxiliary pulley, to the one side of the first reference plane, and the second jaw auxiliary pulley unit (or assembly) may further include a second jaw guide pulley rotatably coupled to the end tool hub and configured to guide the path of the second jaw first wire portion, which extends from the second jaw first auxiliary pulley, to the one side of the first reference plane.

In the other embodiment of the present disclosure, the first jaw auxiliary pulley unit (or assembly) may include a first jaw second-1 auxiliary pulley (or one first jaw second auxiliary pulley) positioned on the one side of the first reference plane and around which the first jaw first wire portion, which extends while being wound around the first jaw coupling pulley, is wound, and a first jaw second-2 auxiliary pulley (or another first jaw second auxiliary pulley) positioned on the another side of the first reference plane and around which the first jaw second wire portion, which extends while being wound around the first jaw coupling pulley, is wound, and the second jaw auxiliary pulley unit (or assembly) may include a second jaw second-1 auxiliary pulley (or one second jaw second auxiliary pulley) positioned on the one side of the first reference plane and around which the second jaw first wire portion, which extends while being wound around the second jaw coupling pulley, is wound, and a second jaw second-2 auxiliary pulley (or another second jaw second auxiliary pulley) positioned on the another side of the first reference plane and around which the second jaw second wire portion, which extends while being wound around the second jaw coupling pulley, is wound.

In the other embodiment of the present disclosure, the first jaw second-1 auxiliary pulley and the second jaw second-1 auxiliary pulley may be rotatably coupled to a first auxiliary shaft positioned in the end tool hub, and the first jaw second-2 auxiliary pulley and the second jaw second-2 auxiliary pulley may be rotatably coupled to a second auxiliary shaft positioned in the end tool hub.

In the other embodiment of the present disclosure, the first jaw second-1 auxiliary pulley and the first jaw second-2 auxiliary pulley may be positioned at different heights from a second reference plane, which is defined as an imaginary plane, so as not to interfere with each other, and the second jaw second-1 auxiliary pulley and the second jaw second-2 auxiliary pulley may be positioned at different heights from the second reference plane so as not to interfere with each other.

In the other embodiment of the present disclosure, the first jaw second-1 auxiliary pulley and the first jaw second-2 auxiliary pulley may be positioned in a tilted arrangement with respect to each other so as not to interfere with each other, and the second jaw second-1 auxiliary pulley and the second jaw second-2 auxiliary pulley may be positioned in a tilted arrangement with respect to each other so as not to interfere with each other.

In an embodiment of the present disclosure, the end tool hub may include a first main guide groove defined on the one side of the first reference plane and configured to guide the path of the first jaw second wire portion extending from the first jaw second-2 auxiliary pulley, and a second main guide groove defined on the another side of the first reference plane and configured to guide the path of the second jaw first wire portion extending from the second jaw second-1 auxiliary pulley.

In the other embodiment of the present disclosure, the first jaw auxiliary pulley unit (or assembly) may further include a first jaw guide pulley rotatably coupled to the end tool hub and configured to guide the path of the first jaw second wire portion, which extends from the first jaw second-2 auxiliary pulley, to the one side of the first reference plane, and the second jaw auxiliary pulley unit (or assembly) may further include a second jaw guide pulley rotatably coupled to the end tool hub and configured to guide the path of the second jaw first wire portion, which extends from the second jaw second-1 auxiliary pulley, to the another side of the first reference plane.

In the other embodiment of the present disclosure, the first jaw auxiliary pulley unit (or assembly) may include a first jaw third auxiliary pulley configured to guide the first jaw first wire portion, which extends from the another side of the first reference plane while being wound around the first jaw coupling pulley, to the one side of the first reference plane, and to guide the first jaw second wire portion, which extends from the one side of the first reference plane while being wound around the first jaw coupling pulley, to the one side of the first reference plane, and the second jaw auxiliary pulley unit (or assembly) may include a second jaw third auxiliary pulley configured to guide the second jaw first wire portion, which extends from the other side of the first reference plane while being wound around the second jaw coupling pulley, to the another side of the first reference plane, and to guide the second jaw second wire portion, which extends from the one side of the first reference plane while being wound around the second jaw coupling pulley, to the another side of the first reference plane.

According to another aspect of the present disclosure, an end tool for a surgical instrument may include a first jaw and a second jaw that are rotatable independently of each other, a first jaw coupling pulley which is coupled to the first jaw and to which a first jaw wire having a first jaw first wire portion and a first jaw second wire portion is coupled, a second jaw coupling pulley which is coupled to the second jaw and to which a second jaw wire having a second jaw first wire portion and a second jaw second wire portion is coupled, and an end tool hub in which the first jaw coupling pulley and the second jaw coupling pulley are rotatably positioned, the first jaw wire is guided to one side of a first reference plane, which is defined as an imaginary plane, and the second jaw wire is guided to another side of the first reference plane, where a path of a first jaw first wire portion and a path of a first jaw second wire portion are adjusted to intersect or cross each other, and a path of a second jaw first wire portion and a path of a second jaw second wire portion are adjusted to intersect or cross each other.

In the other embodiment of the present disclosure, the end tool hub may include a hub main body, and a hub guide body extending from the hub main body and interconnecting a pair of hub extensions that are facing each other, where the hub guide body may include a first-1 auxiliary guide groove (or one first auxiliary guide groove) configured to guide the path of the first jaw first wire portion, which extends from the another side of the first reference plane while being wound around the first jaw coupling pulley, to the one side of the first reference plane, a first-2 auxiliary guide groove (or another first auxiliary guide groove) configured to guide the path of the first jaw second wire portion, which extends from the one side of the first reference plane while being wound around the first jaw coupling pulley, to the another side of the first reference plane, a second-1 auxiliary guide groove (or one second auxiliary guide groove) configured to guide the path of the second jaw first wire portion, which extends from the another side of the first reference plane while being wound around the second jaw coupling pulley, to the one side of the first reference plane, and a second-2 auxiliary guide groove (another second auxiliary guide groove) configured to guide the path of the second jaw second wire portion, which extends from the one side of the first reference plane while being wound around the second jaw coupling pulley, to the another side of the first reference plane.

In the other embodiment of the present disclosure, the first-1 auxiliary guide groove and the first-2 auxiliary guide groove may be positioned at different heights from a second reference plane, which is defined as an imaginary plane, and the second-1 auxiliary guide groove and the second-2 auxiliary guide groove may be positioned at different heights from the second reference plane.

In the other embodiment of the present disclosure, the hub main body may include a first main guide groove defined on the one side of the first reference plane and configured to guide the path of the first jaw second wire portion extending from the first-2 auxiliary guide groove, and a second main guide groove defined on the another side of the first reference plane and configured to guide the path of the second jaw first wire portion extending from the second-1 auxiliary guide groove.

In the other embodiment of the present disclosure, the end tool may further include a first jaw guide pulley rotatably coupled to the hub main body and configured to guide the path of the first jaw second wire portion, which extends from the first-2 auxiliary guide groove, to the one side of the first reference plane, and a second jaw guide pulley rotatably coupled to the hub main body and configured to guide the path of the second jaw first wire portion, which extends from the second-1 auxiliary guide groove, to the another side of the first reference plane.

According to the other aspect of the present disclosure, an end tool for a surgical instrument may include a first jaw and a second jaw that are rotatable independently of each other, a first jaw coupling pulley which is coupled to the first jaw and to which a first jaw wire having a first jaw first wire portion and a first jaw second wire portion is coupled, a second jaw coupling pulley which is coupled to the second jaw and to which a second jaw wire having a second jaw first wire portion and a second jaw second wire portion is coupled, an end tool hub in which the first jaw coupling pulley and the second jaw coupling pulley are rotatably positioned, and a wire guide unit (or portion) configured to adjust paths of the first jaw first wire portion and the first jaw second wire portion within the end tool hub so as to guide the first jaw wire to extend to an outside of the end tool hub from one side of a first reference plane, which is defined as an imaginary plane, and to adjust paths of the second jaw first wire portion and the second jaw second wire portion within the end tool hub so as to guide the second jaw wire to extend to the outside of the end tool hub from another side of the first reference plane.

In the other embodiment of the present disclosure, the wire guide unit may include a first jaw guide unit configured to adjust the paths of the first jaw first wire portion and the first jaw second wire portion within the end tool hub to intersect each other, and a second jaw guide unit configured to adjust the paths of the second jaw first wire portion and the second jaw second wire portion within the end tool hub to intersect each other.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1A is a conceptual view of a pitch motion of a conventional surgical instrument, and FIG. 1B is a conceptual view of a yaw motion.

FIG. 1C is a conceptual view of a pitch motion of another conventional surgical instrument, and FIG. 1D is a conceptual view of a yaw motion.

FIG. 1E is a conceptual view of a pitch motion of a surgical instrument according to the present disclosure, and FIG. 1F is a conceptual view of a yaw motion.

FIG. 2 is a perspective view illustrating a surgical instrument according to an embodiment of the present disclosure.

FIG. 3 is a side view of the surgical instrument of FIG. 2.

FIGS. 4 and 5 are perspective views illustrating an end tool of the surgical instrument according to an embodiment of the present disclosure.

FIG. 6 is a perspective view illustrating an end tool hub of the end tool of the surgical instrument of FIG. 4.

FIG. 7 is a plan view illustrating an end tool of a conventional surgical instrument.

FIG. 8 is a plan view illustrating the end tool of the surgical instrument of FIG. 4.

FIG. 9 is a conceptual view illustrating a wire guide structure of the end tool of the surgical instrument of FIG. 4.

FIGS. 10 and 11 are perspective views illustrating an end tool of a surgical instrument according to another embodiment of the present disclosure.

FIG. 12 is a plan view illustrating the end tool of the surgical instrument of FIG. 10.

FIGS. 13 and 14 are conceptual views illustrating a wire guide structure of the end tool of the surgical instrument of FIG. 10.

FIGS. 15 to 17 are views illustrating modified examples of the wire guide structure of the end tool of the surgical instrument of FIG. 13.

FIGS. 18 and 19 are perspective views illustrating an end tool of a surgical instrument according to the other embodiment of the present disclosure.

FIG. 20 is a plan view illustrating the end tool of the surgical instrument of FIG. 18.

FIG. 21 is a perspective view illustrating some components of the end tool of the surgical instrument of FIG. 18.

FIG. 22 is a conceptual view illustrating a wire guide structure of the end tool of the surgical instrument of FIG. 18.

FIGS. 23 and 24 are perspective views illustrating an end tool of a surgical instrument according to the other embodiment of the present disclosure.

FIG. 25 is a perspective view illustrating an end tool hub of the end tool of the surgical instrument of FIG. 23.

FIG. 26 is a conceptual view illustrating a wire guide structure of the end tool of the surgical instrument of FIG. 23.

FIG. 27 is a view illustrating a modified example of the wire guide structure of the end tool of the surgical instrument of FIG. 23.

FIGS. 28 and 29 are views illustrating a manipulation part of the surgical instrument of FIG. 2.

FIG. 30 is a view schematically illustrating only a configuration of the pulleys and wires constituting joints of the surgical instrument according to an embodiment of the present disclosure illustrated in FIG. 28.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” “third,” “fourth,” and the like may be used to describe various components, such components should not be limited to the above terms. The terms are only used to distinguish one component from another.

In the following embodiments, terms such as "include" or "have" means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as "connecting" or "coupling" two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

In a surgical instrument according to the present disclosure, when a manipulation part is rotated in one direction for at least any one of pitch, yaw, and actuation motions, an end tool is rotated in intuitively the same direction as a direction in which the manipulation part is manipulated.

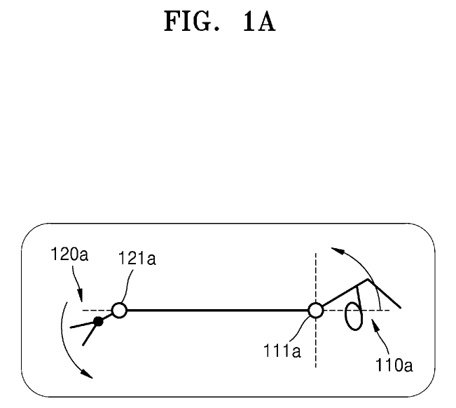

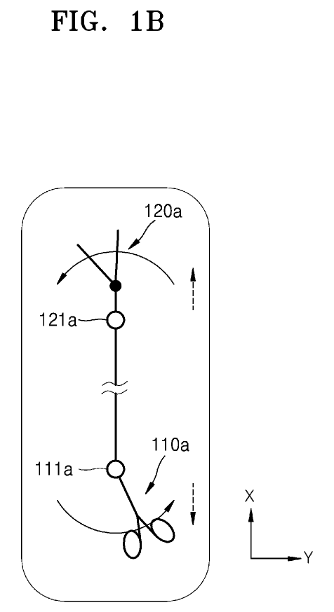

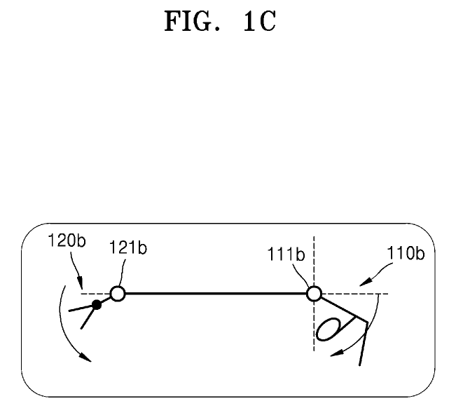

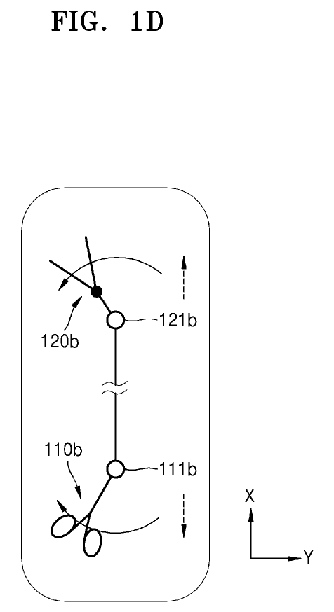

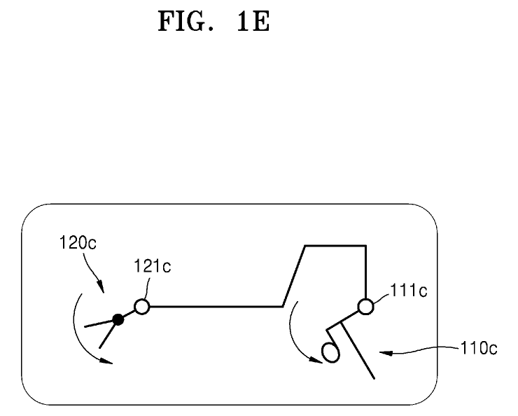

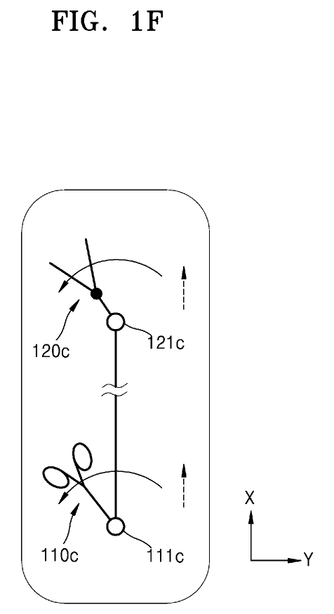

FIG. 1A is a conceptual view of a pitch motion of a conventional surgical instrument, and FIG. 1B is a conceptual view of a yaw motion. FIG. 1C is a conceptual view of a pitch motion of another conventional surgical instrument, and FIG. 1D is a conceptual view of a yaw motion. FIG. 1E is a conceptual view of a pitch motion of a surgical instrument according to the present disclosure, and FIG. 1F is a conceptual view of a yaw motion.

Referring to FIG. 1A, in performing a pitch motion of a conventional surgical instrument, in a state in which an end tool 120a is formed in front of a rotation center 121a of the end tool, and a manipulation part 110a is formed at the rear of a rotation center 111a of the manipulation part, when the manipulation part 110a is rotated in a clockwise direction, the end tool 120a is also rotated in the clockwise direction, and when the manipulation part 110a is rotated in a counterclockwise direction, the end tool 120a is also rotated in the counterclockwise direction.

Referring to FIG. 1B, in performing a yaw motion of the conventional surgical instrument, in a state in which the end tool 120a is formed in front of the rotation center 121a of the end tool, and the manipulation part 110a is formed at the rear of the rotation center 111a of the manipulation part, when the manipulation part 110a is rotated in the clockwise direction, the end tool 120a is also rotated in the clockwise direction, and when the manipulation part 110a is rotated in the counterclockwise direction, the end tool 120a is also rotated in the counterclockwise direction. In this case, in view of left and right directions of a user, when the user moves the manipulation part 110a to the left, the end tool 120a is moved to the right, and when the user moves the manipulation part 110a to the right, the end tool 120a is moved to the left. As a result, a manipulation direction of the user and an operation direction of the end tool are opposite to each other, which may cause the user to make a mistake, and user's manipulation may not be easy.

Referring to FIG. 1C, in the conventional surgical instrument, which is partially formed in a mirror symmetrical shape, in performing a pitch motion, in a state in which an end tool 120b is formed in front of a rotation center 121b of the end tool, and a manipulation part 110b is formed at the rear of a rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error.

Referring to FIG. 1D, in performing a yaw motion, in a state in which the end tool 120b is formed in front of the rotation center 121b of the end tool, and the manipulation part 110b is formed at the rear of the rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error.

In the user's pitch or yaw manipulation of the conventional surgical instrument, the user's manipulation direction and the end tool's operation direction do not match each other in view of one of the rotation direction and the left and right directions. This is because the configurations of the end tool and the manipulation part are different from each other in the joint configuration of the conventional surgical instrument. That is, this is because the manipulation part is formed at the rear of the rotation center of the manipulation part, while the end tool is formed in front of the rotation center of the end tool.

In order to address the above problems, in a surgical instrument according to an embodiment of the present disclosure, which is illustrated of FIGS. 1E and 1F, an end tool 120c is formed in front of a rotation center 121c of the end tool and a manipulation part 110c is also formed in front of a rotation center 111c of the manipulation part, so that the operations of the manipulation part 110c and the end tool 120c are intuitively matched with each other. In other words, unlike existing examples such as those shown in FIGS. 1A, 1B, 1C, and FIG. 1D, in which the manipulation part is close to a user with respect to the joint thereof that is, away from the end tool, the surgical instrument according to an embodiment of the present disclosure, which is illustrated of FIGS. 1E and 1F, is formed such that at least a portion of the manipulation part is closer than a joint thereof to the end tool with respect to the joint thereof at any one moment or more in a manipulation process.

In other words, in the conventional surgical instrument as illustrated of FIGS. 1A, 1B, 1C, and FIG. 1D, the manipulation part is formed at the rear of the rotation center thereof, while the end tool is positioned in front of the rotation center thereof, and thus the end tool is moved at a front side thereof with a rear side fixed through a motion of the manipulation part that is moved at a rear side thereof with a front side thereof fixed, which is not an intuitively matching structure. Accordingly, a mismatch may occur between the manipulation of the manipulation part and the motion of the end tool in view of the left and right directions or in view of the rotation direction, which may cause confusion to the user, and the manipulation, operation, or control of the manipulation part may be difficult to perform intuitively and quickly and may cause mistakes.

In contrast, in the surgical instrument according to an embodiment of the present disclosure, since both the end tool and the manipulation part are moved with respect to the rotation center formed at the rear side thereof, it may be said that the motions are intuitively matched with each other in terms of structure. In other words, moving portions of the manipulation part are moved with respect to the rotation center formed at the rear side thereof just as moving portions of the end tool are moved with respect to the rotation center formed at the rear side thereof, and thus it may be said that the motions are intuitively matched with each other in terms of structure. This allows the user to intuitively and quickly perform a control in a direction toward the end tool, and a possibility of making a mistake may be significantly reduced. Hereinafter, a detailed mechanism enabling the above-described function will be described.

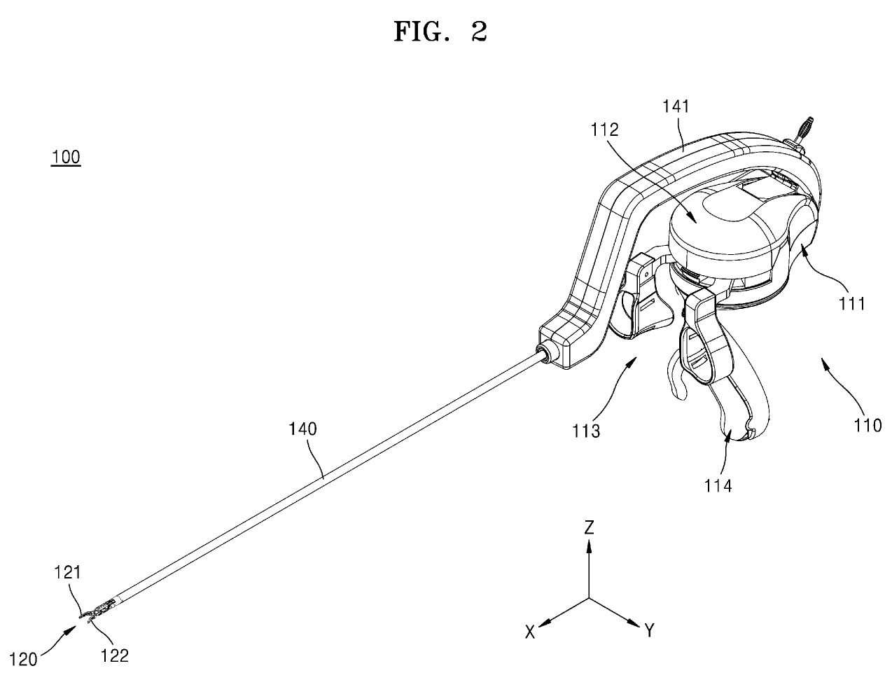

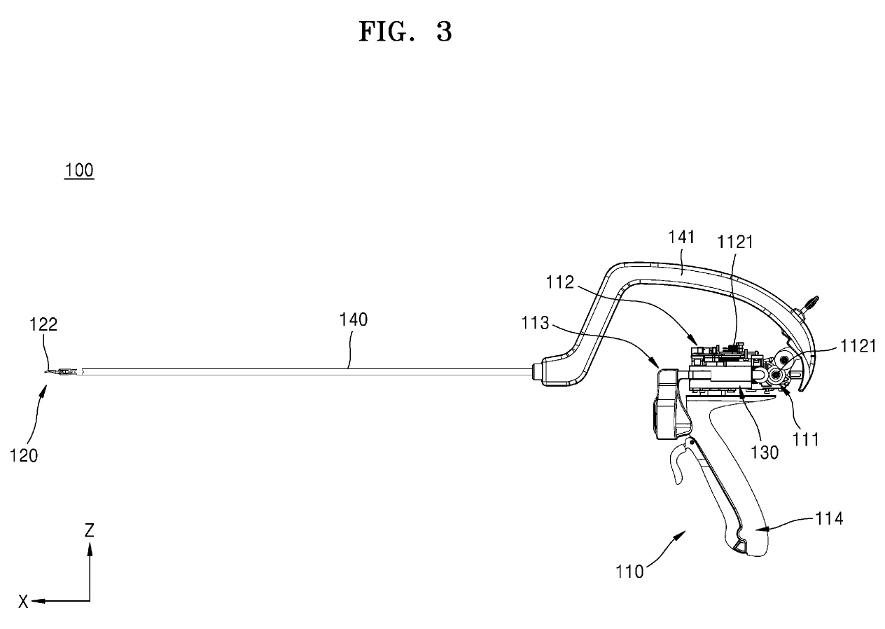

FIG. 2 is a perspective view illustrating a surgical instrument 100 according to an embodiment of the present disclosure, and FIG. 3 is a side view of the surgical instrument 100 of FIG. 2.

Referring to FIGS. 2 and 3, the surgical instrument 100 according to an embodiment of the present disclosure may include a manipulation part 110, an end tool 120, a power transmission part 130, and a connection part 140. Here, the connection part 140 is formed in the shape of a hollow shaft, in which one or more wires to be described later may be accommodated, and may have one end portion to which the manipulation part 110 is coupled and another end portion to which the end tool 120 is coupled and serve to connect the manipulation part 110 to the end tool 120. Here, the connection part 140 of the surgical instrument 100 according to an embodiment of the present disclosure has a bent portion 141 formed on the side of the manipulation part 110. As such, since the end portion of the connection part 140 at the side of the manipulation part 110 is configured to be bent, a pitch manipulation part 111, a yaw manipulation part 112, and an actuation manipulation part 113 may be formed along an extension line of the end tool 120 or adjacent to the extension line. In other words, it may be said that the pitch manipulation part 111 and the yaw manipulation part 112 are at least partially accommodated in a concave portion formed by the bent portion 141. Due to the above-described shape of the bent portion 141, the shapes and motions of the manipulation part 110 and the end tool 120 may be further intuitively matched with each other.

A plane on which the bent portion 141 is formed may be substantially the same as a pitch plane, that is, an XZ plane of FIG. 2. As such, as the bent portion 141 is formed on the plane substantially the same as the XZ plane, interference with the manipulation part may be reduced. Of course, for intuitive motions of the end tool and the manipulation part, configurations in planes other than the XZ plane may also be possible.

The manipulation part 110 is formed on one end portion of the connection part 140 and provided as an interface to be directly controlled by a medical doctor, for example, a tongs shape, a stick shape, a lever shape, or the like, and when the medical doctor controls the manipulation part 110, the end tool 120, which is connected to the interface and inserted into the body of a surgical patient, performs a certain motion, thereby performing surgery. Here, the manipulation part 110 is illustrated in FIG. 2 as being formed in a handle shape that is rotatable while the finger is inserted therein, but the concept of the present disclosure is not limited thereto, and various types of manipulation parts that are connected to the end tool 120 and manipulate the end tool 120 may be possible.

The end tool 120 is formed on another end portion of the connection part 140, and performs necessary motions for surgery by being inserted into a surgical site. In an example of the above-described end tool 120, as shown in FIG. 2, a pair of jaws 121 and 122 for performing a grip motion may be used. However, the concept of the present disclosure is not limited thereto, and various devices for performing surgery may be used as the end tool 120. For example, a configuration such as a cantilever cautery may also be used as the end tool. The end tool 120 is connected to the manipulation part 110 by the power transmission part 130, and receives a driving force of the manipulation part 110 through the power transmission part 130 to perform a motion necessary for surgery, such as gripping, cutting, suturing, or the like.

Here, the end tool 120 of the surgical instrument 100 according to an embodiment of the present disclosure is configured to be rotatable in at least one direction, for example, the end tool 120 may perform a pitch motion around a Y-axis of FIG. 2 and simultaneously perform a yaw motion and an actuation motion around a Z-axis of FIG. 2.

Here, each of the pitch, yaw, and actuation motions used in the present disclosure are defined as follows.

First, the pitch motion means a motion of the end tool 120 rotating in a vertical direction with respect to an extension direction of the connection part 140 (an X-axis direction of FIG. 2), that is, a motion rotating around the Y-axis of FIG. 2. In other words, the pitch motion means a motion of the end tool 120, which is configured to extend from the connection part 140 in the extension direction of the connection part 140 (the X-axis direction of FIG. 2), rotating vertically around the Y-axis with respect to the connection part 140. Next, the yaw motion means a motion of the end tool 120 rotating in left and right directions, that is, a motion rotating around the Z-axis of FIG. 2, with respect to the extension direction of the connection part 140 (the X-axis direction of FIG. 2). In other words, the yaw motion means a motion of the end tool 120, which is configured to extend from the connection part 140 in the extension direction of the connection part 140 (the X-axis direction of FIG. 2), rotating horizontally around the Z-axis with respect to the connection part 140. That is, the yaw motion means a motion of two jaws 121 and 122, which are formed on the end tool 120, rotating around the Z-axis in the same direction. In an embodiment, the actuation motion means a motion of the end tool 120 rotating around the same shaft of rotation as that of the yaw motion, while the two jaws 121 and 122 rotate in opposite directions so as to be closed or opened. That is, the actuation motion means a motion of the two jaws 121 and 122, which are formed on the end tool 120, rotating in the opposite directions around the Z-axis.

The power transmission part 130 may serve to connect the manipulation part 110 to the end tool 120 to transmit the driving force of the manipulation part 110 to the end tool 120, and may include a plurality of wires, pulleys, links, sections, gears, and the like. In the surgical instrument 100 according to an embodiment of the present disclosure, the power transmission part 130 may include a pitch wire 130P, a first jaw wire JW1, and a second jaw wire JW2.

Hereinafter, the manipulation part 110, the end tool 120, the power transmission part 130, and the like of the surgical instrument 100 of FIG. 2 will be described in more detail.

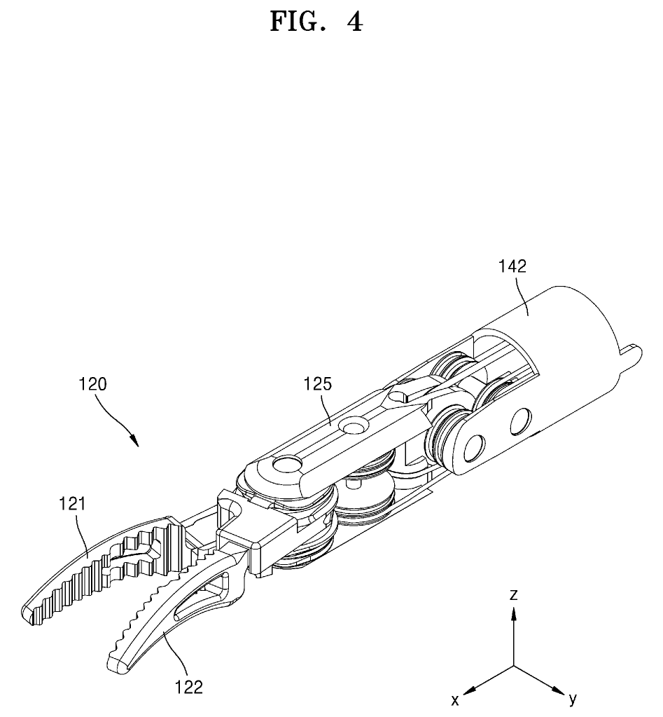

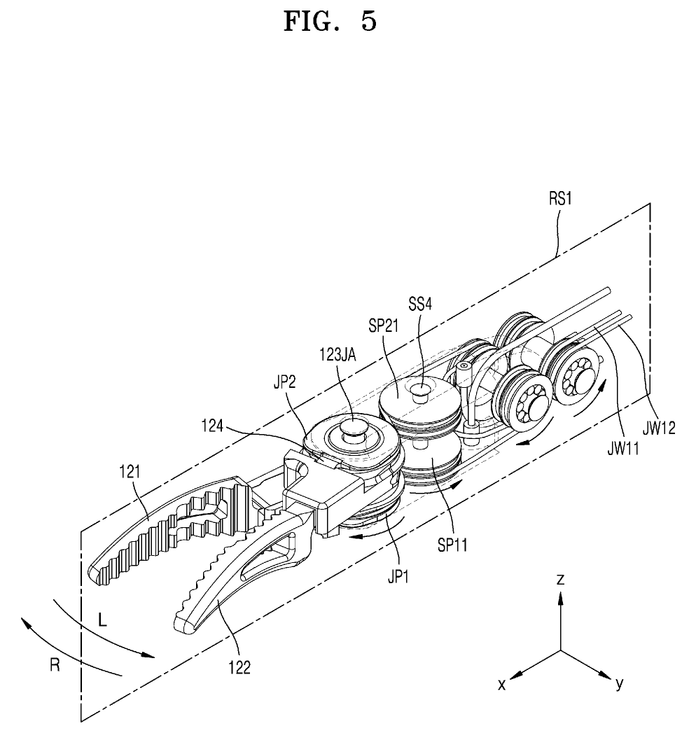

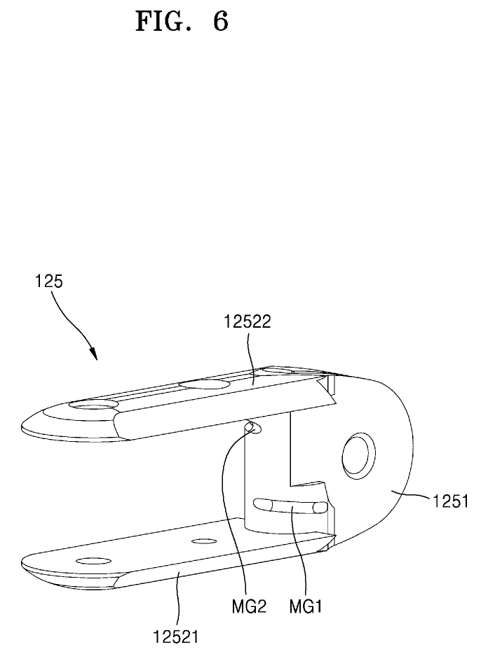

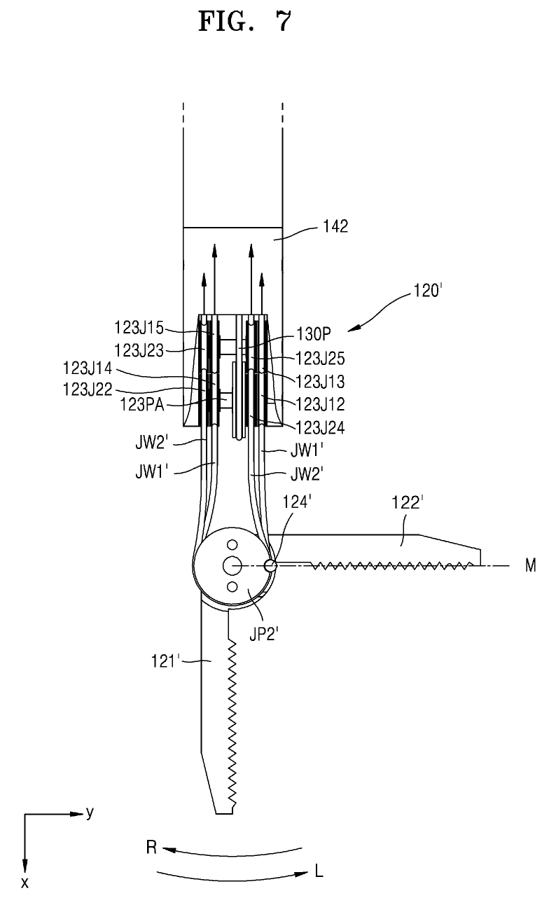

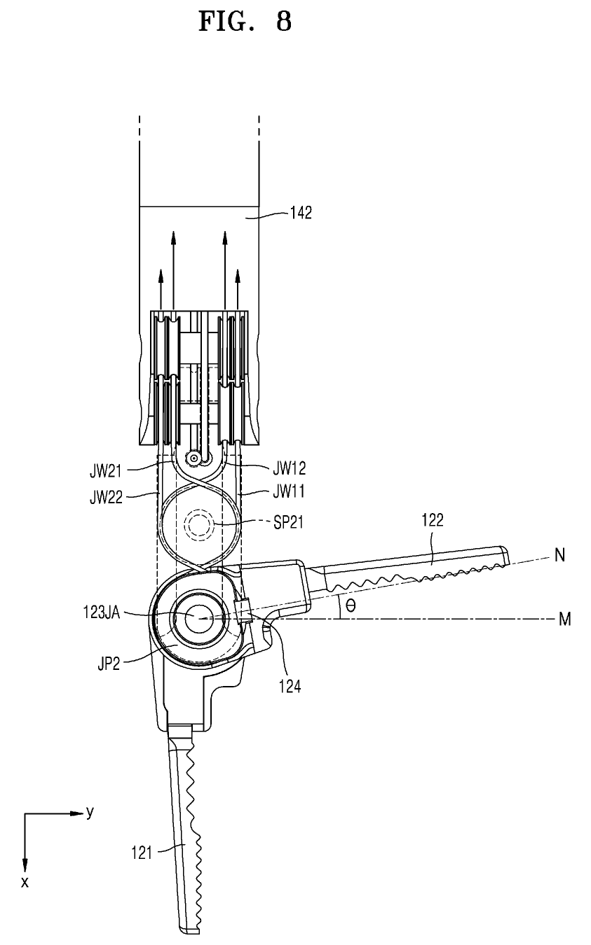

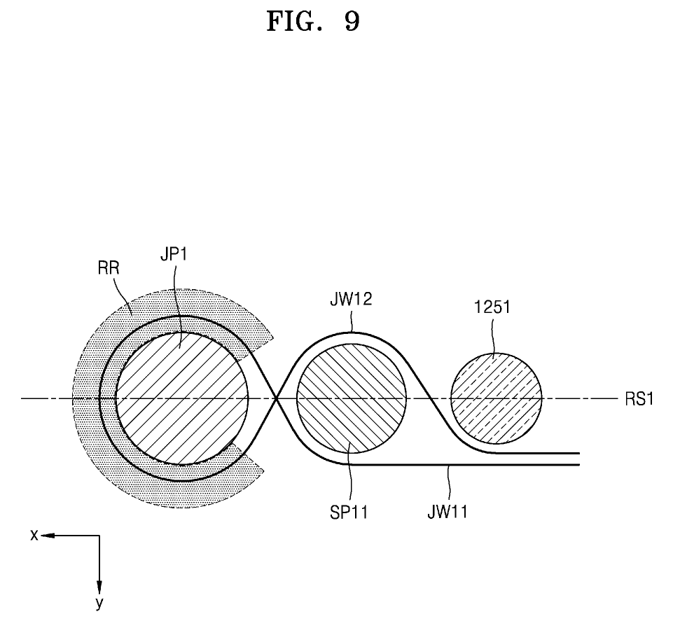

FIGS. 4 and 5 are perspective views illustrating the end tool 120 of the surgical instrument according to an embodiment of the present disclosure, and FIG. 6 is a perspective view illustrating an end tool hub 125 of the end tool of the surgical instrument of FIG. 4. In addition, FIG. 7 is a plan view illustrating an end tool 120' of a conventional surgical instrument, and FIG. 8 is a plan view illustrating the end tool 120 of the surgical instrument of FIG. 4. In addition, FIG. 9 is a conceptual view illustrating a wire guide structure of the end tool 120 of the surgical instrument of FIG. 4.

First, referring to FIGS. 4, 5, 6, and 8, the end tool 120 according to an embodiment of the present disclosure includes the pair of jaws 121 and 122, i.e., a first jaw 121 and a second jaw 122, for performing a grip motion. The end tool 120 may further include a plurality of pulleys associated with rotational movements of the first jaw 121 and the second jaw 122. The end tool 120 may further include the end tool hub 125. Here, the first jaw 121, the second jaw 122, and the plurality of pulleys may all be configured to rotate together around an end tool pitch rotation shaft 123PA.

The end tool 120 may include the first jaw wire JW1, the second jaw wire JW2, and the pitch wire 130P. The first jaw wire JW1 and the second jaw wire JW2 may control rotational movements of the first jaw 121 and the second jaw 122, respectively. For example, the first jaw 121 and the second jaw 122 may perform a pitch rotation around the end tool pitch rotation shaft 123PA using the pitch wire 130P.

The first jaw wire JW1 may include a first jaw first wire portion JW11 and a first jaw second wire portion JW12. The first jaw first wire portion JW11 and the first jaw second wire portion JW12 may be implemented as a single wire. In the present specification, a coupling member (not shown), which is a first jaw wire-end tool coupling member, may be inserted at an intermediate point of the single first jaw wire JW1, and then a coupling member 124 may be fixed by crimping. Two strands of the first jaw wire JW1 extending from opposite sides of the coupling member (not shown) may be referred to as the first jaw first wire portion JW11 and the first jaw second wire portion JW12, respectively.

Alternatively, the first jaw first wire portion JW11 and the first jaw second wire portion JW12 may be formed as separate wires and connected to each other by a coupling member (not shown). For example, the coupling member (not shown) may be coupled to a first jaw coupling pulley JP1, which will be described later, so that the first jaw first wire portion JW11 and the first jaw second wire portion JW12 are fixedly coupled to the first jaw coupling pulley JP1. Accordingly, the first jaw coupling pulley JP1 may rotate as the first jaw first wire portion JW11 and the first jaw second wire portion JW12 are pulled and released.

In some embodiments, opposite end portions of the first jaw first wire portion JW11 and the first jaw second wire portion JW12, which are opposite to portions fastened to the coupling member 124, may be coupled to first jaw wire-manipulation part coupling members (not shown).

For example, as the first jaw wire-manipulation part coupling members (not shown) may each be coupled to a pulley 210 as described above, the first jaw first wire portion JW11 and the first jaw second wire portion JW12 are fixedly coupled to the pulley 210. As a result, when the pulley 210 is rotated by a motor or manual force, the first jaw coupling pulley JP1 may be rotated as the first jaw first wire portion JW11 and the first jaw second wire portion JW12 are pulled and released.

In the same manner, a second jaw first wire portion JW21 and a second jaw second wire portion JW22 of the second jaw wire JW2 may be coupled to a second jaw wire-end tool coupling member 124 and a second jaw wire-manipulation part coupling member (not shown), respectively. For example, the coupling member 124 may be coupled to a second jaw coupling pulley JP2, and the second jaw wire-manipulation part coupling member (not shown) may be coupled to a pulley 220. As a result, when the pulley 220 is rotated by a motor or manual force, the second jaw coupling pulley JP2 may be rotated as the second jaw first wire portion JW21 and the second jaw second wire portion JW22 are pulled and released.

As a result, the two strands of the first jaw wire JW1, that is, the first jaw first wire portion JW11 and the first jaw second wire portion JW12, may be coupled to the first jaw wire-end tool coupling member (not shown) and the first jaw wire-manipulation part coupling member (not shown), respectively, and may be configured to function as a closed loop as a whole. Similarly, the second jaw wire JW2 may also be configured to function as a closed loop.

The first jaw wire JW1 and the second jaw wire JW2 may be positioned on at least one of the plurality of pulleys provided in the end tool 120. The pulleys included in the end tool 120 will now be described in detail as follows.

First, the end tool 120 may include the first jaw coupling pulley JP1 and the second jaw coupling pulley JP2.

The first jaw coupling pulley JP1 may be coupled to the first jaw 121, and the first jaw first wire portion JW11 and the first jaw second wire portion JW12 may be positioned thereon. The first jaw first wire portion JW11 and the first jaw second wire portion JW12 may be coupled to the first jaw coupling pulley JP1 using the coupling member 124, and may control rotational movement of the first jaw 121.

The second jaw coupling pulley JP2 may be coupled to the second jaw 122, and the second jaw first wire portion JW21 and the second jaw second wire portion JW22 may be positioned thereon. The second jaw first wire portion JW21 and the second jaw second wire portion JW22 may be coupled to the second jaw coupling pulley JP2 using the coupling member 124, and may control rotational movement of the second jaw 122.

The first jaw coupling pulley JP1 and the second jaw coupling pulley JP2 are configured to face each other and to rotate independently around a jaw rotation shaft 123JA. Here, the first jaw 121 may be coupled to the first jaw coupling pulley JP1 to rotate together therewith, and the second jaw 122 may be coupled to the second jaw coupling pulley JP2 to rotate together therewith. Yaw and actuation motions of the end tool 120 are performed according to the rotation of the first jaw coupling pulley JP1 and the second jaw coupling pulley JP2. That is, when the first jaw coupling pulley JP1 and the second jaw coupling pulley JP2 rotate in the same direction, the yaw motion is performed, and when the first jaw coupling pulley JP1 and the second jaw coupling pulley JP2 rotate in opposite directions, the actuation motion is performed.

The end tool 120 may further include a J12 pulley 123J12, a J13 pulley 123J13, a J14 pulley 123J14, a J15 pulley 123J15, a J22 pulley 123J22, a J23 pulley 123J23, a J24 pulley 123J24, and a J25 pulley 123J25.

A connection part hub 142 is positioned at one end portion of the connection part 140 coupled to the end tool 120. The J12 pulley 123J12, the J13 pulley 123J13, the J14 pulley 123J14, the J15 pulley 123J15, the J22 pulley 123J22, the J23 pulley 123J23, the J24 pulley 123J24, and the J25 pulley 123J25 may be coupled to the connection part hub 142. The first jaw wire JW1 and the second jaw wire JW2 may be positioned on at least one of the above-described pulleys and may extend to the shaft of the connection part 140 via the connection part hub 142.

For example, the end tool 120 may further include a pitch pulley 123P, around which the pitch wire 130P is wound. The pitch pulley 123P may rotate around the end tool pitch rotation shaft 123PA to control a pitch rotation of the end tool 120.

Here, although the facing pulleys are illustrated as being formed in parallel to each other in the drawings, the concept of the present disclosure is not limited thereto, and each pulley may be formed in various positions and sizes suitable for the configuration of the end tool.

The end tool 120 may further include the end tool hub 125 that serves as a base of the end tool 120 and is configured to allow coupling of the first jaw coupling pulley JP1, the second jaw coupling pulley JP2, and the pitch pulley 123P.

The end tool hub 125 may include a hub main body 1251 and a pair of hub extensions 12521 and 12522 extending from the hub main body 1251. The end tool 120 may be shaft-coupled to the connection part hub 142 using the hub main body 1251, and the first jaw coupling pulley JP1 and the second jaw coupling pulley JP2 may be disposed between the pair of hub extensions 12521 and 12522 and shaft-coupled to the end tool hub 125.

In some embodiments, referring to FIG. 7, in the conventional end tool 120', since a first jaw wire JW1′ and a second jaw wire JW2′ are fixedly coupled to a first jaw coupling pulley (not shown) and a second jaw coupling pulley JP2′, respectively, the first jaw coupling pulley (not shown) and the second jaw coupling pulley JP2′ may each rotate only up to line M in the direction of arrow L in FIG. 7. In other words, the first jaw coupling pulley (not shown) and the second jaw coupling pulley JP2′ may be allowed to rotate only up to an approximately perpendicular angle beyond which the first jaw wire JW1′ and a coupling member 124′ would become separated.

In this case, when an actuation operation is performed while a first jaw 121′ and a second jaw 122′ are positioned on line M of FIG. 7, the first jaw 121′ may move further in the direction of arrow R, but the second jaw 122′ may not rotate beyond line M in the direction of arrow L. Accordingly, when the first jaw 121′ and the second jaw 122′ are performing a yaw motion beyond a certain angle, an actuation motion may not be smoothly performed.

In some embodiments, the end tool 120 of the present disclosure may include a plurality of auxiliary pulleys or guide grooves capable of functioning as auxiliary pulleys to control the paths of the first jaw wire JW1 and the second jaw wire JW2. In other words, the end tool 120 may include a wire guide unit in the form of the end tool hub 125 having auxiliary pulleys or guide grooves functioning as auxiliary pulleys, and accordingly, opening and closing radii of the first jaw 121 and the second jaw 122 may be adjusted. Hereinafter, the driving of the first jaw 121 and the second jaw 122 will be described with a focus on various embodiments of the end tool 120 having auxiliary pulleys and/or guide grooves.

First, in the present specification, an imaginary plane that is formed at the center of the end tool 120, is perpendicular to the Y-axis of FIG. 5, and passes through the X-axis is defined as a first reference plane RS1. The first jaw second wire portion JW12 may be wound around the first jaw coupling pulley JP1 in a region on one side, i.e., in a +Y direction, with respect to the first reference plane RS1. In some embodiments, the first jaw first wire portion JW11 may be wound around the first jaw coupling pulley JP1 in a region on another side, i.e., in a -Y direction, with respect to the first reference plane RS1.

Similarly, the second jaw second wire portion JW22 may be wound around the second jaw coupling pulley JP2 in a region on one side, i.e., in the +Y direction, with respect to the first reference plane RS1. In some embodiments, the second jaw first wire portion JW21 may be wound around the second jaw coupling pulley JP2 in a region on another side, i.e., in the –Y direction, with respect to the first reference plane RS1. The end tool 120 of the present disclosure may include a wire guide unit in the form of the end tool hub 125 having auxiliary pulleys and/or guide grooves, and may adjust the paths of respective wire portions based on one side and another side of the first reference plane RS1.

Referring again to FIGS. 4, 5, 6, 8, and FIG. 9, the end tool 120 according to an embodiment of the present disclosure may further include a first jaw auxiliary pulley unit and a second jaw auxiliary pulley unit. The first jaw auxiliary pulley unit and the second jaw auxiliary pulley unit may adjust paths of the first jaw wire JW1 and the second jaw wire JW2, respectively, thereby enabling adjustment of opening and closing radii of the first jaw 121 and the second jaw 122.

The first jaw wire JW1 extending from the first jaw coupling pulley JP1 may be wound along the first jaw auxiliary pulley unit. The first jaw auxiliary pulley unit may include a plurality of first jaw auxiliary pulleys SP1, and the first jaw first wire portion JW11 and the first jaw second wire portion JW12 of the first jaw wire JW1 may extend while being wound around at least one of the plurality of first jaw auxiliary pulleys SP1. That is, the first jaw auxiliary pulley unit may guide the paths of the first jaw first wire portion JW11 and the first jaw second wire portion JW12 extending from the first jaw coupling pulley JP1.

The first jaw auxiliary pulley unit may include a first jaw first auxiliary pulley SP11. The first jaw first auxiliary pulley SP11 may adjust the paths of the first jaw first wire portion JW11 and the first jaw second wire portion JW12.

For example, the first jaw first auxiliary pulley SP11 may be rotatably coupled to a fourth auxiliary shaft SS4 to adjust the path of the first jaw wire JW1. The first jaw first auxiliary pulley SP11 may guide the first jaw first wire portion JW11, which extends from another side of the first reference plane RS1 while being wound around the first jaw coupling pulley JP1, to one side of the first reference plane RS1. For example, the first jaw first auxiliary pulley SP11 may guide the first jaw second wire portion JW12, which extends from one side of the first reference plane RS1 while being wound around the first jaw coupling pulley JP1, to another side of the first reference plane RS1. That is, the first jaw first auxiliary pulley SP11 may adjust the paths of both the first jaw first wire portion JW11 and the first jaw second wire portion JW12, and in particular, may cause the paths of the first jaw first wire portion JW11 and the first jaw second wire portion JW12 extending from the first jaw coupling pulley JP1 to intersect each other.

The second jaw wire JW2 extending from the second jaw coupling pulley JP2 may be wound on the second jaw auxiliary pulley unit. The second jaw auxiliary pulley unit may include a plurality of second jaw auxiliary pulleys SP2, and the second jaw first wire portion JW21 and the second jaw second wire portion JW22 of the second jaw wire JW2 may extend while being wound around at least one of the plurality of second jaw auxiliary pulleys SP2. That is, the second jaw auxiliary pulley unit may guide paths of the second jaw first wire portion JW21 and the second jaw second wire portion JW22 extending from the second jaw coupling pulley JP2.

The second jaw auxiliary pulley unit may include a second jaw first auxiliary pulley SP21. The second jaw first auxiliary pulley SP21 may adjust the paths of the second jaw first wire portion JW21 and the second jaw second wire portion JW22.

For example, the second jaw first auxiliary pulley SP21 may guide the second jaw first wire portion JW21, which extends from another side of the first reference plane RS1 while being wound around the second jaw coupling pulley JP2, to one side of the first reference plane RS1. The second jaw first auxiliary pulley SP21 may guide the second jaw second wire portion JW22, which extends from one side of the first reference plane RS1 while being wound around the second jaw coupling pulley JP2, to another side of the first reference plane RS1. That is, the second jaw first auxiliary pulley SP21 may adjust the paths of both the second jaw first wire portion JW21 and the second jaw second wire portion JW22, and in particular, may allow the paths of the second jaw first wire portion JW21 and the second jaw second wire portion JW22 extending from the second jaw coupling pulley JP2 to intersect each other.

The end tool hub 125 of the end tool 120 may include a plurality of main guide grooves MG for adjusting the paths of the first jaw wire JW1 and the second jaw wire JW2. The main guide grooves MG may be formed as recessed grooves in the hub main body 1251 to guide the paths of the first jaw wire JW1 and the second jaw wire JW2, which respectively extend from the first jaw auxiliary pulley unit and the second jaw auxiliary pulley unit. The first jaw wire JW1 and the second jaw wire JW2 may be wound around the main guide grooves MG of the end tool hub 125 and extend through the end tool hub 125 to the shaft of the connection part 140.

The end tool hub 125 may include a first main guide groove MG1 and a second main guide groove MG2.

The first main guide groove MG1 may guide the first jaw second wire portion JW12, which extends from another side of the first reference plane RS1 while being wound around the first jaw first auxiliary pulley SP11, to one side of the first reference plane RS1. That is, by the first main guide groove MG1, the first jaw first wire portion JW11 and the first jaw second wire portion JW12 may extend from one side of the first reference plane RS1 together to the connection part 140.

The second main guide groove MG2 may guide the second jaw first wire portion JW21, which extends from one side of the first reference plane RS1 while being wound around the second jaw first auxiliary pulley SP21, to another side of the first reference plane RS1. That is, by the second main guide groove MG2, the second jaw first wire portion JW21 and the second jaw second wire portion JW22 may extend from another side of the first reference plane RS1 together to the connection part 140.

The rotation of the first jaw 121 and the second jaw 122 according to the arrangement of the paths of the first jaw wire JW1 and the second jaw wire JW2 is summarized as follows.

The first jaw first wire portion JW11 may extend from another side of the first reference plane RS1 while being wound around the first jaw coupling pulley JP1, and a path thereof may be adjusted by the first jaw first auxiliary pulley SP11 to one side of the first reference plane RS1 and then may extend to the connection part 140. The first jaw second wire portion JW12 may extend from one side of the first reference plane RS1 while being wound around the first jaw coupling pulley JP1, and a path thereof may be adjusted by the first jaw first auxiliary pulley SP11 to another side of the first reference plane RS1. The first jaw second wire portion JW12 may extend from another side of the first reference plane RS1 while being wound around the first jaw first auxiliary pulley SP11, and a path thereof may be adjusted by the first main guide groove MG1 to one side of the first reference plane RS1 and then may extend to the connection part 140.

Accordingly, the end tool 120 may change both tangential directions of two strands of the first jaw wire JW1 when the two strands extend from the first jaw coupling pulley JP1. The first jaw wire portions JW11 and JW12, together with a coupling member (not shown), may be rotatable until the first jaw wire portions JW11 and JW12 are positioned on a common internal tangent of the first jaw coupling pulley JP1 and the first jaw first auxiliary pulley SP11. Thus, when the first jaw 121 rotates, rotation radii RR in both clockwise and counterclockwise directions about the jaw rotation shaft 123JA may be increased.

The second jaw first wire portion JW21 may extend from another side of the first reference plane RS1 while being wound around the second jaw coupling pulley JP2, and a path thereof may be adjusted by the second jaw first auxiliary pulley SP21 to one side of the first reference plane RS1. The second jaw first wire portion JW21 may extend from one side of the first reference plane RS1 while being wound around the second jaw first auxiliary pulley SP21, and a path thereof may be adjusted by the second main guide groove MG2 to another side of the first reference plane RS1 and then may extend to the connection part 140. The second jaw second wire portion JW22 may extend from one side of the first reference plane RS1 while being wound around the second jaw coupling pulley JP2, and a path thereof may be adjusted by the second jaw first auxiliary pulley SP21 to another side of the first reference plane RS1 and then may extend to the connection part 140.

Accordingly, when two strands of the second jaw wire JW2 extend from the second jaw coupling pulley JP2, the end tool 120 is able to change both of their tangential directions. The second jaw wires portions JW21 and JW22, together with a coupling member (not shown), may be rotatable until the second jaw wires portions JW21 and JW22 are positioned on a common internal tangent of the second jaw coupling pulley JP2 and the second jaw first auxiliary pulley SP21. Thus, when the second jaw 122 rotates, the rotation radii RR in both the clockwise and counterclockwise directions about the jaw rotation shaft 123JA may be increased.

That is, even when the first jaw 121 and the second jaw 122 are positioned on line M in FIG. 8, the first jaw 121 may be rotatable, and the second jaw 122 may also be additionally rotatable by an angle θ up to line N in FIG. 8. In the opposite situation, the first jaw 121 may also be rotatable, thereby increasing a yaw motion range in which an actuation motion can be performed.

The rotation radii of the first jaw 121 and the second jaw 122 may be determined according to the directions in which the first jaw wire JW1 and the second jaw wire JW2 extend from the first jaw coupling pulley JP1 and the second jaw coupling pulley JP2, respectively. Accordingly, in a variation of the embodiment shown in FIG. 9, the first jaw auxiliary pulley unit of the end tool 120 may include a first jaw third auxiliary pulley (not shown) that guides the first jaw first wire portion JW11, which extends from another side of the first reference plane RS1 while being wound around the first jaw coupling pulley JP1, to one side of the first reference plane RS1, and guides the first jaw second wire portion JW12, which extends from one side of the first reference plane RS1 while being wound around the first jaw coupling pulley JP1, to the one side of the first reference plane RS1.

In some embodiments, the second jaw auxiliary pulley unit of the end tool 120 may include a second jaw third auxiliary pulley (not shown) that guides the second jaw first wire portion JW21, which extends from another side of the first reference plane RS1 while being wound around the second jaw coupling pulley JP2, to the other side of the first reference plane RS1, and guides the second jaw second wire portion JW22, which extends from one side of the first reference plane RS1 while being wound around the second jaw coupling pulley JP2, to another side of the first reference plane RS1.

As described above, in the end tool 120 according to an embodiment of the present disclosure, the opening and closing radii may be increased by adjusting the paths of the first jaw wire JW1 and the second jaw wire JW2 through the first jaw first auxiliary pulley SP11, the second jaw first auxiliary pulley SP21, the first main guide groove MG1, and the second main guide groove MG2 of the end tool hub 125.

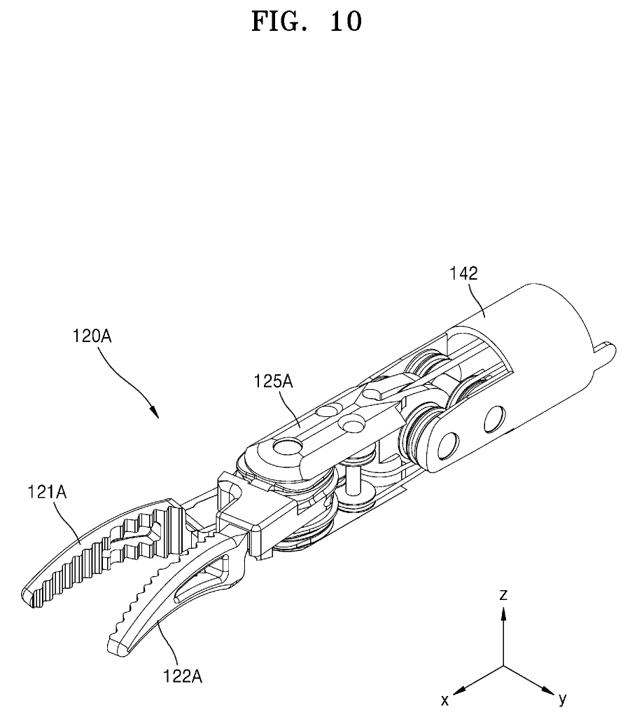

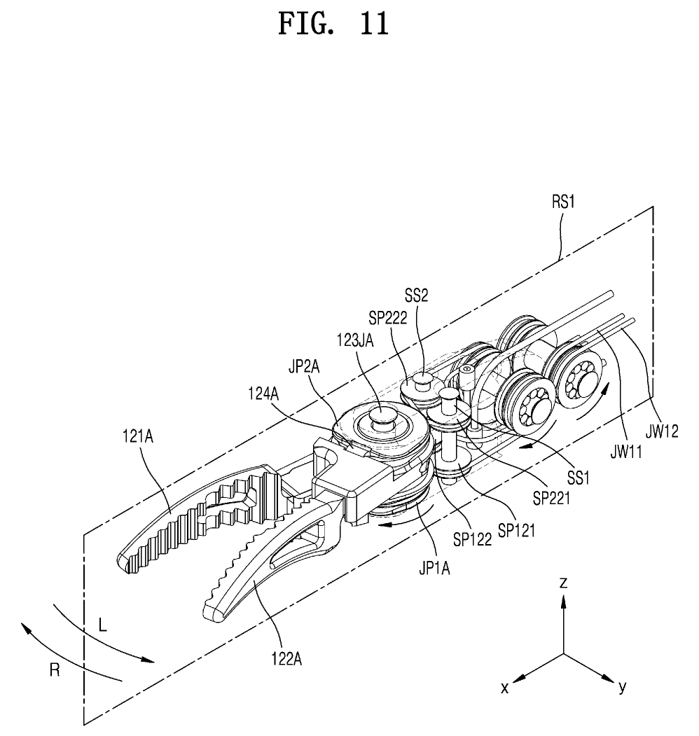

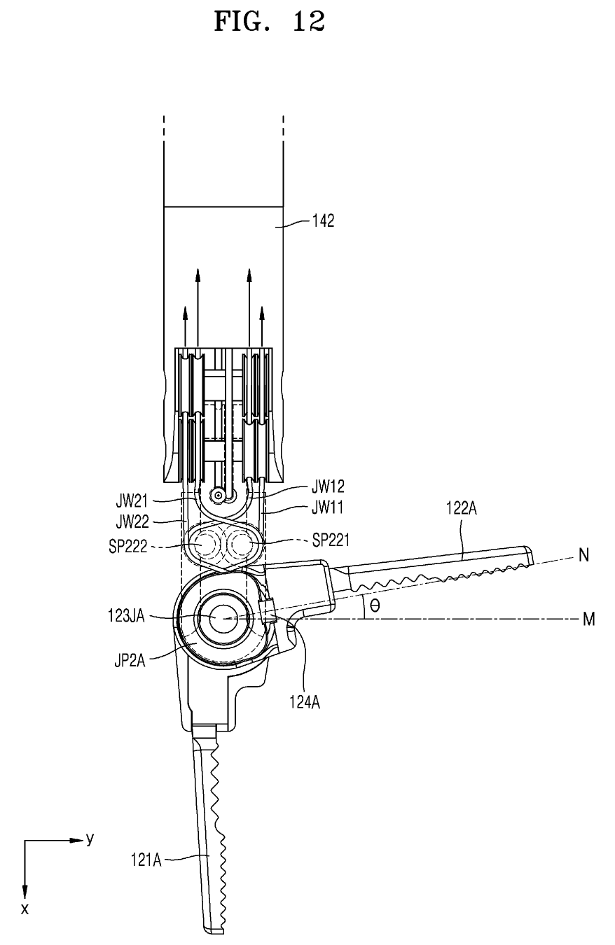

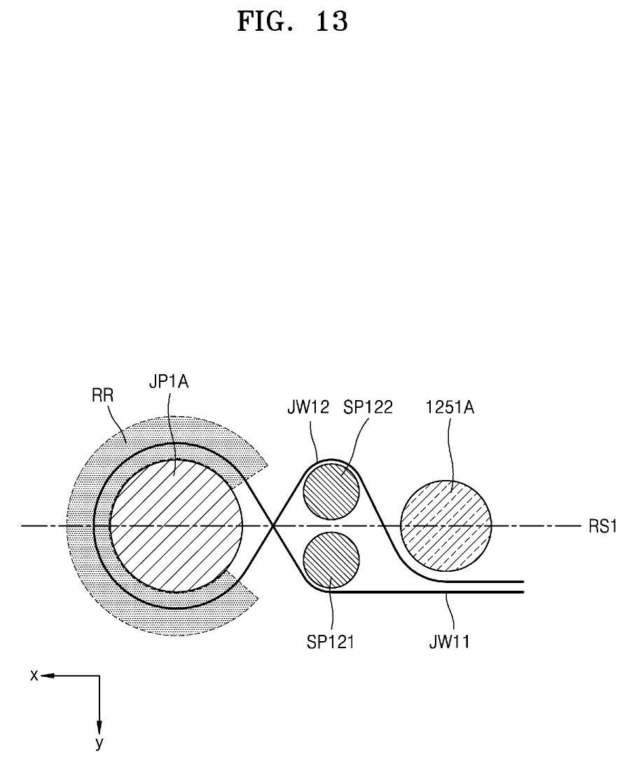

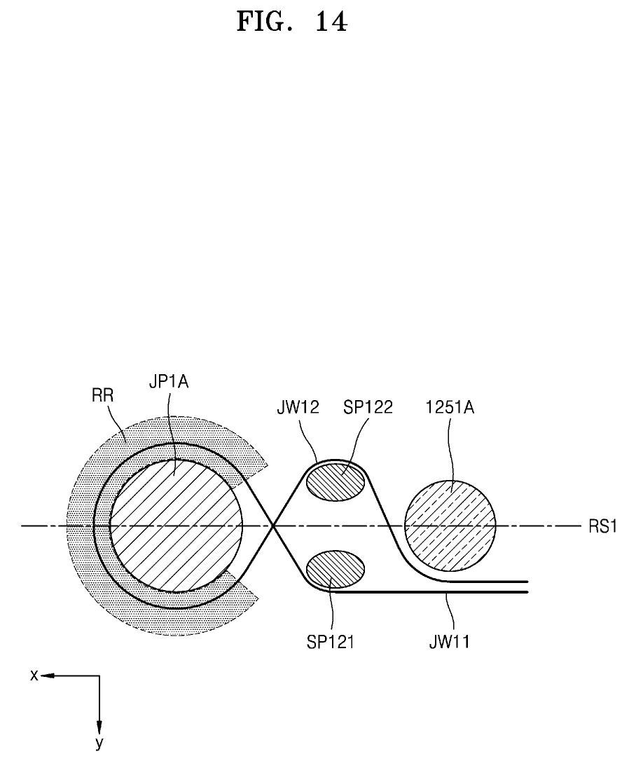

FIGS. 10 and 11 are perspective views illustrating an end tool 120A of a surgical instrument according to another embodiment of the present disclosure, and FIG. 12 is a plan view illustrating the end tool 120A of the surgical instrument of FIG. 10. FIGS. 13 and 14 are conceptual views illustrating a wire guide structure of the end tool 120A of the surgical instrument of FIG. 10. FIGS. 15 to 17 are views illustrating modified examples of the wire guide structure of the end tool 120A of the surgical instrument of FIG. 13.

The end tool 120A of the surgical instrument according to another embodiment of the present disclosure differs in the configuration and arrangement of a wire guide unit including auxiliary pulleys. Accordingly, the following description focuses on these differences, and the descriptions of the other components may be referred to FIG. 4 and the like as previously described.

Referring to FIGS. 10 to 13, the end tool 120A according to another embodiment of the present disclosure may include a first jaw auxiliary pulley unit and a second jaw auxiliary pulley unit. The first jaw auxiliary pulley unit and the second jaw auxiliary pulley unit may adjust paths of a first jaw wire JW1 and a second jaw wire JW2, respectively, thereby enabling adjustment of opening and closing radii of a first jaw 121A and a second jaw 122A.

The first jaw wire JW1 extending from a first jaw coupling pulley JP1A may be wound on the first jaw auxiliary pulley unit. The first jaw auxiliary pulley unit may include a plurality of first jaw auxiliary pulleys SP1, and the first jaw first wire portion JW11 and the first jaw second wire portion JW12 of the first jaw wire JW1 may extend while being wound around at least one of the plurality of first jaw auxiliary pulleys SP1. That is, the first jaw auxiliary pulley unit may guide paths of the first jaw first wire portion JW11 and the first jaw second wire portion JW12 extending from the first jaw coupling pulley JP1A.

The first jaw auxiliary pulley unit may include a first jaw second-1 auxiliary pulley SP121 and a first jaw second-2 auxiliary pulley SP122. The first jaw second-1 auxiliary pulley SP121 and the first jaw second-2 auxiliary pulley SP122 may adjust the paths of the first jaw first wire portion JW11 and the first jaw second wire portion JW12, respectively.

For example, the first jaw second-1 auxiliary pulley SP121 may be rotatably coupled to a first auxiliary shaft SS1 and may adjust the path of the first jaw first wire portion JW11. The first jaw second-1 auxiliary pulley SP121 may guide the first jaw first wire portion JW11, which extends from another side of a first reference plane RS1 while being wound around the first jaw coupling pulley JP1A, to one side of the first reference plane RS1.

The first jaw second-2 auxiliary pulley SP122 may be rotatably coupled to a second auxiliary shaft SS2 and may adjust the path of the first jaw second wire portion JW12. The first jaw second-2 auxiliary pulley SP122 may guide the first jaw second wire portion JW12, which extends from one side of the first reference plane RS1 while being wound around the first jaw coupling pulley JP1A, to another side of the first reference plane RS1. That is, the paths of the first jaw first wire portion JW11 and the first jaw second wire portion JW12, both extending from the first jaw coupling pulley JP1A, may intersect by the first jaw second-1 auxiliary pulley SP121 and the first jaw second-2 auxiliary pulley SP122.

The second jaw wire JW2 extending from a second jaw coupling pulley JP2A may be wound on the second jaw auxiliary pulley unit. The second jaw auxiliary pulley unit may include a plurality of second jaw auxiliary pulleys SP2, and the second jaw first wire portion JW21 and the second jaw second wire portion JW22 of the second jaw wire JW2 may extend while being wound around at least one of the plurality of second jaw auxiliary pulleys SP2. That is, the second jaw auxiliary pulley unit may guide paths of the second jaw first wire portion JW21 and the second jaw second wire portion JW22 extending from the second jaw coupling pulley JP2.

The second jaw auxiliary pulley unit may include a second jaw second-1 auxiliary pulley SP221 and a second jaw second-2 auxiliary pulley SP222. The second jaw second-1 auxiliary pulley SP221 and the second jaw second-2 auxiliary pulley SP222 may adjust the paths of the second jaw first wire portion JW21 and the second jaw second wire portion JW22, respectively.

For example, the second jaw second-1 auxiliary pulley SP221 may be rotatably coupled to the first auxiliary shaft SS1 and may adjust the path of the second jaw first wire portion JW21. The second jaw second-1 auxiliary pulley SP221 may guide the second jaw first wire portion JW21, which extends from another side of the first reference plane RS1 while being wound around the second jaw coupling pulley JP2A, to one side of the first reference plane RS1.

The second jaw second-2 auxiliary pulley SP222 may be rotatably coupled to the second auxiliary shaft SS2 and may adjust the path of the second jaw second wire portion JW22. The second jaw second-2 auxiliary pulley SP222 may guide the second jaw second wire portion JW22, which extends from one side of the first reference plane RS1 while being wound around the second jaw coupling pulley JP2A, to another side of the first reference plane RS1. That is, the paths of the second jaw first wire portion JW21 and the second jaw second wire portion JW22, both extending from the second jaw coupling pulley JP2A, may intersect by the second jaw second-1 auxiliary pulley SP221 and the second jaw second-2 auxiliary pulley SP222.

That is, the first jaw second-1 auxiliary pulley SP121 and the second jaw second-1 auxiliary pulley SP221 may be rotatably coupled to the first auxiliary shaft SS1, and the first jaw second-2 auxiliary pulley SP122 and the second jaw second-2 auxiliary pulley SP222 may be rotatably coupled to the second auxiliary shaft SS2. In this case, the first jaw second-1 auxiliary pulley SP121 and the first jaw second-2 auxiliary pulley SP122 may be positioned at the same height with respect to an imaginary second reference plane (not shown) parallel to an XY plane. Similarly, the second jaw second-1 auxiliary pulley SP221 and the second jaw second-2 auxiliary pulley SP222 may be positioned at the same height with respect to the second reference plane (not shown).

In some embodiments, the first jaw second-1 auxiliary pulley SP121 and the first jaw second-2 auxiliary pulley SP122 may be positioned at different heights with respect to the second reference plane (not shown), and the second jaw second-1 auxiliary pulley SP221 and the second jaw second-2 auxiliary pulley SP222 may also be positioned at different heights with respect to the second reference plane (not shown).

For example, the first jaw second-1 auxiliary pulley SP121 and the first jaw second-2 auxiliary pulley SP122 may be disposed in parallel with the imaginary second reference plane (not shown) parallel to the XY plane. Similarly, the second jaw second-1 auxiliary pulley SP221 and the second jaw second-2 auxiliary pulley SP222 may be positioned parallel to the second reference plane (not shown).

As shown in FIG. 14, the first jaw second-1 auxiliary pulley SP121 and the first jaw second-2 auxiliary pulley SP122 may be tilted at a predetermined angle with respect to the second reference plane (not shown), and the second jaw second-1 auxiliary pulley SP221 and the second jaw second-2 auxiliary pulley SP222 may also be tilted at a predetermined angle with respect to the second reference plane (not shown).

In this manner, the auxiliary pulleys disposed inside the end tool hub 125 may guide the paths of the jaw wires JW1 and JW2 efficiently without interference with each other by being positioned at different heights or angles, thereby allowing the end tool 120 to be miniaturized and reduced in weight.

An end tool hub 125A may include a first main guide groove MG1 and a second main guide groove MG2.

The first main guide groove MG1 may guide the first jaw second wire portion JW12, which extends from another side of the first reference plane RS1 while being wound around the first jaw second-2 auxiliary pulley SP122, to one side of the first reference plane RS1. That is, by the first main guide groove MG1, the first jaw first wire portion JW11 and the first jaw second wire portion JW12 may extend from one side of the first reference plane together to the connection part 140.

The second main guide groove MG2 may guide the second jaw first wire portion JW21, which extends from one side of the first reference plane RS1 while being wound around the second jaw second-1 auxiliary pulley SP221, to another side of the first reference plane RS1. That is, by the second main guide groove MG2, the second jaw first wire portion JW21 and the second jaw second wire portion JW22 may extend from another side of the first reference plane RS1 together to the connection part 140.

The rotation of the first jaw 121A and the second jaw 122A according to the arrangement of the paths of the first jaw wire JW1 and the second jaw wire JW2 is summarized as follows.

The first jaw first wire portion JW11 may extend from another side of the first reference plane RS1 while being wound around the first jaw coupling pulley JP1A, and a path thereof may be adjusted by the first jaw second-1 auxiliary pulley SP121 to one side of the first reference plane RS1 and then may extend to the connection part 140. The first jaw second wire portion JW12 may extend from one side of the first reference plane RS1 while being wound around the first jaw coupling pulley JP1A, and a path thereof may be adjusted by the first jaw second-2 auxiliary pulley SP122 to another side of the first reference plane RS1. The first jaw second wire portion JW12 may extend from another side of the first reference plane RS1 while being wound around the first jaw second-1 auxiliary pulley SP121, and a path thereof may be adjusted by the first main guide groove MG1 to one side of the first reference plane RS1 and then may extend to the connection part 140.

Accordingly, the end tool 120A may change both tangential directions of two strands of the first jaw wire JW1 when the two strands extend from the first jaw coupling pulley JP1A. The first jaw wire portions JW11 and JW12 and a coupling member (not shown) may be rotatable until positioned on a common internal tangent of the first jaw coupling pulley JP1A and the first jaw second-1 auxiliary pulley SP121, and may also be rotatable until positioned on a common internal tangent of the first jaw coupling pulley JP1A and the first jaw second-2 auxiliary pulley SP122. Thus, when the first jaw 121A rotates, rotation radii RR in both the clockwise and counterclockwise directions about a jaw rotation shaft 123JA may be increased.

The second jaw first wire portion JW21 may extend from another side of the first reference plane RS1 while being wound around the second jaw coupling pulley JP2A, and a path thereof may be adjusted by the second jaw second-1 auxiliary pulley SP221 to one side of the first reference plane RS1. The second jaw first wire portion JW21 may extend from one side of the first reference plane RS1 while being wound around the second jaw second-1 auxiliary pulley SP221, and a path thereof may be adjusted by the second main guide groove MG2 to another side of the first reference plane RS1 and then may extend toward the connection part 140. The second jaw second wire portion JW22 may extend from one side of the first reference plane RS1 while being wound around the second jaw coupling pulley JP2A, and a path thereof may be adjusted by the second jaw second-1 auxiliary pulley SP221 to another side of the first reference plane RS1 and then may extend toward the connection part 140.

Accordingly, when two strands of the second jaw wire JW2 extend from the second jaw coupling pulley JP2A, the end tool 120A is able to change both of their tangential directions. The second jaw wire portions JW21 and JW22 and a coupling member (not shown) may be rotatable until positioned on a common internal tangent of the second jaw coupling pulley JP2A and the second jaw second-1 auxiliary pulley SP221, and may also be rotatable until positioned on a common internal tangent of the second jaw coupling pulley JP2A and the second jaw second-2 auxiliary pulley SP222. Thus, when the second jaw 122A rotates, the rotation radii RR in both the clockwise and counterclockwise directions about the jaw rotation shaft 123JA may be increased.

That is, even when the first jaw 121A and the second jaw 122A are positioned on line M in FIG. 12, the first jaw 121A may be rotatable, and the second jaw 122A may also be additionally rotatable by an angle θ up to line N in FIG. 12. In the opposite situation, the first jaw 121A may also be rotatable, thereby increasing a yaw motion range in which an actuation motion can be performed.

As described above, in the end tool 120A according to an embodiment of the present disclosure, opening and closing radii may be increased by adjusting the paths of the first jaw wire JW1 and the second jaw wire JW2 through the first jaw second-1 auxiliary pulley SP121, the first jaw second-2 auxiliary pulley SP122, the second jaw second-1 auxiliary pulley SP221, the second jaw second-2 auxiliary pulley SP222, the first main guide groove MG1, and the second main guide groove MG2 of the end tool hub 125A.

FIGS. 15 to 17 illustrate modified examples in which the wire guide unit adjusts the paths of the first jaw wire JW1 and the second jaw wire JW2 differently. As described above, the rotation radii of the first jaw 121A and the second jaw 122A may be determined according to the directions in which the first jaw wire JW1 and the second jaw wire JW2 extend from the first jaw coupling pulley JP1A and the second jaw coupling pulley JP2A, respectively. Accordingly, as shown in FIGS. 15 to 17, by changing the arrangement of the paths of the first jaw wire JW1 and the second jaw wire JW2 of the end tool 120A, the rotation radii of the first jaw 121A and the second jaw 122A may be adjusted. Hereinafter, with reference to FIGS. 15 to 17, the description will focus on the arrangement of the path of the first jaw wire JW1, while the path arrangement of the second jaw wire JW2 may also be adjusted in the same manner.

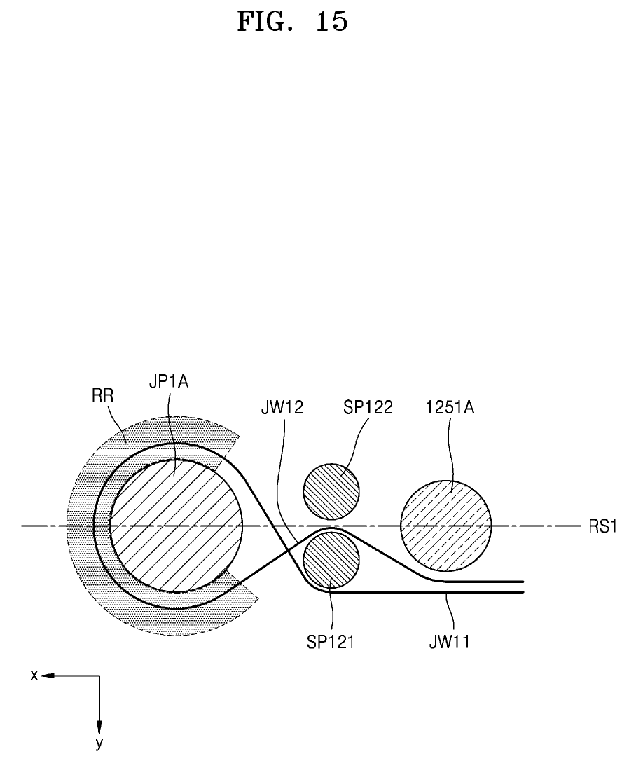

For example, as shown in FIG. 15, the first jaw second wire portion JW12 may extend from one side of the first reference plane RS1 while being wound around the first jaw coupling pulley JP1A and may also be wound around the first jaw second-1 auxiliary pulley SP121, which is located on one side of and adjacent to the first reference plane RS1, to extend to the one side of the first reference plane RS1. Alternatively, as shown in FIG. 16, the first jaw first wire portion JW11 may extend from another side of the first reference plane RS1 while being wound around the first jaw coupling pulley JP1A and may also be wound around the first jaw second-1 auxiliary pulley SP121, which is positioned on one side of and adjacent to the first reference plane RS1, to extend to the one side of the first reference plane RS1.

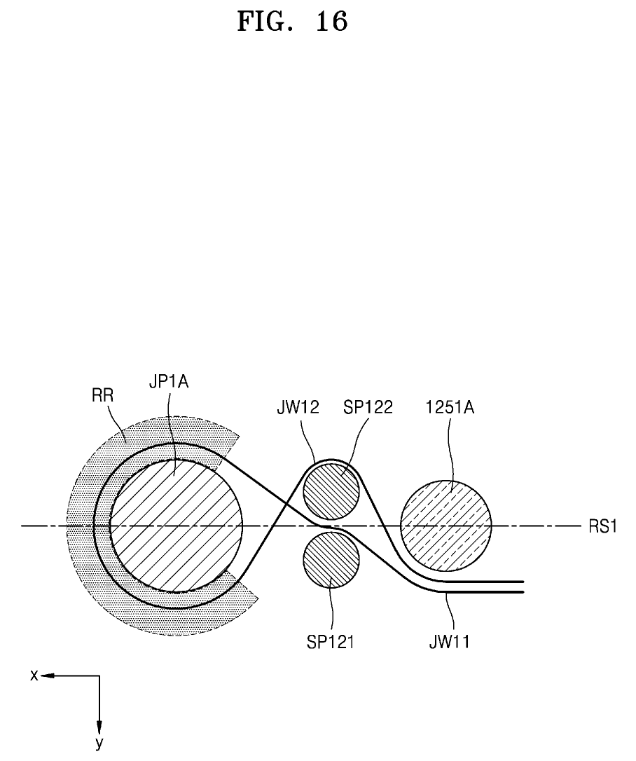

Alternatively, as shown in FIG. 17, the first jaw first wire portion JW11 may extend from another side of the first reference plane RS1 while being wound around the first jaw coupling pulley JP1A, and may also be wound around the first jaw second-2 auxiliary pulley SP122, which is positioned on one side of and adjacent to the first reference plane RS1, to extend to the one side of the first reference plane RS1. The first jaw second wire portion JW12 may extend from one side of the first reference plane RS1 while being wound around the first jaw coupling pulley JP1A, and may also be wound around the first jaw second-1 auxiliary pulley SP121, which is positioned on one side of and adjacent to the first reference plane RS1, to extend to the one side of the first reference plane RS1.

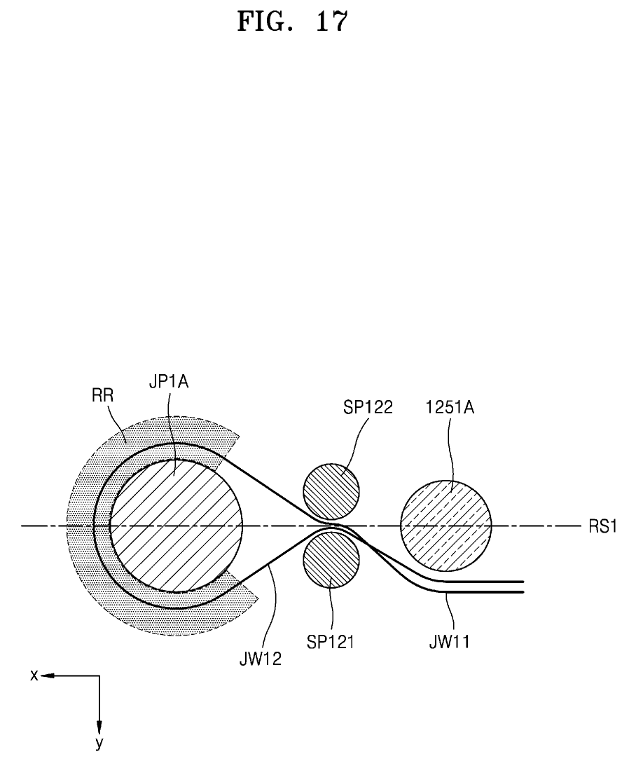

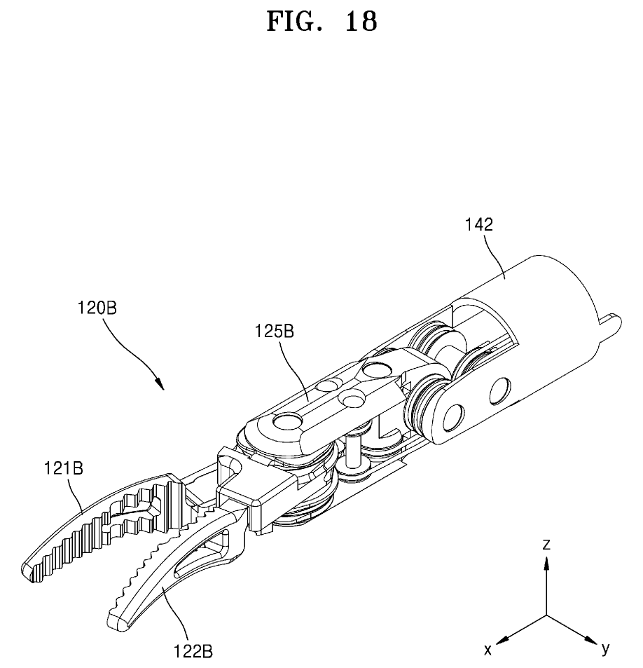

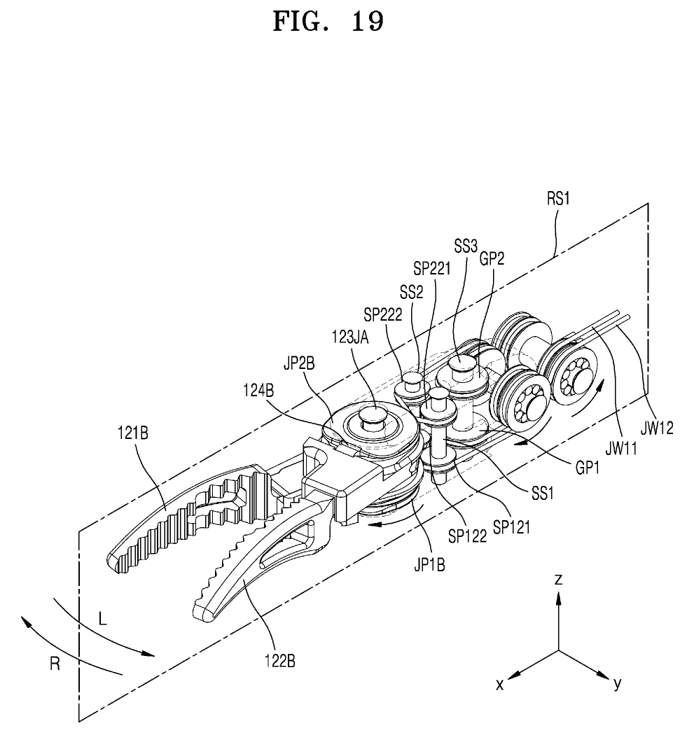

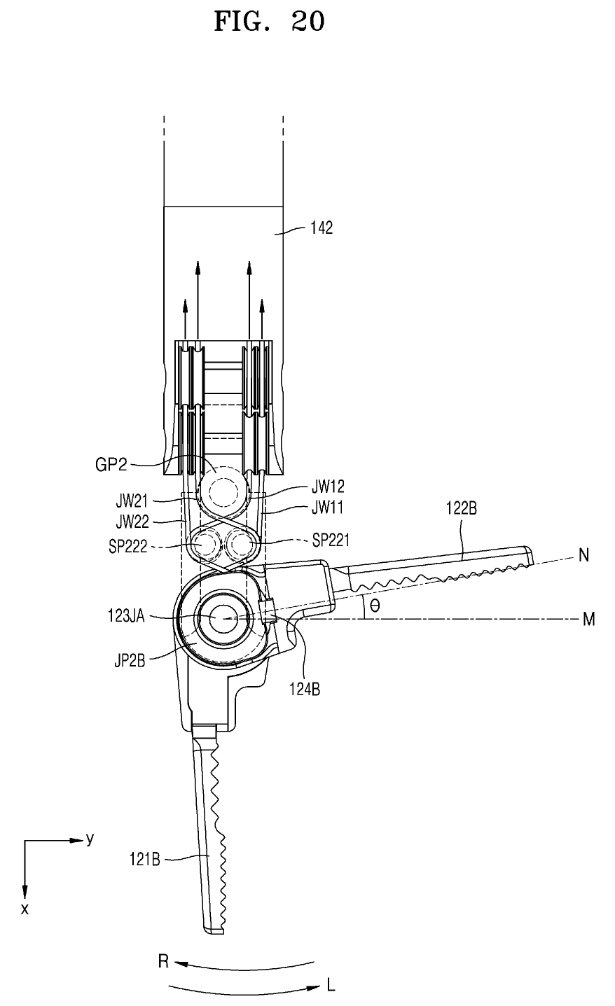

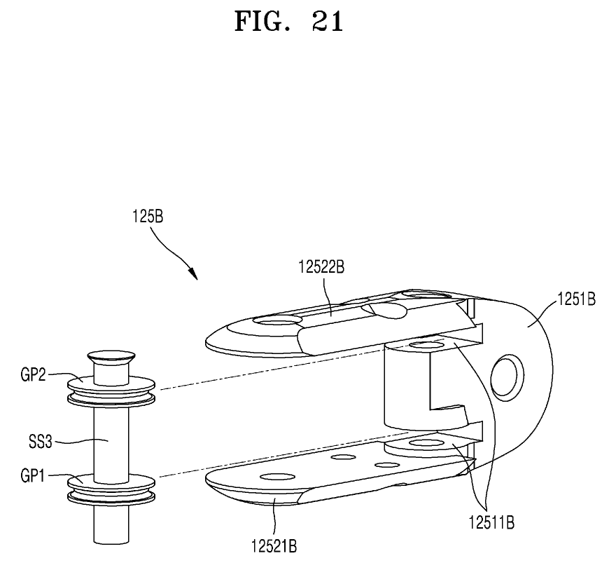

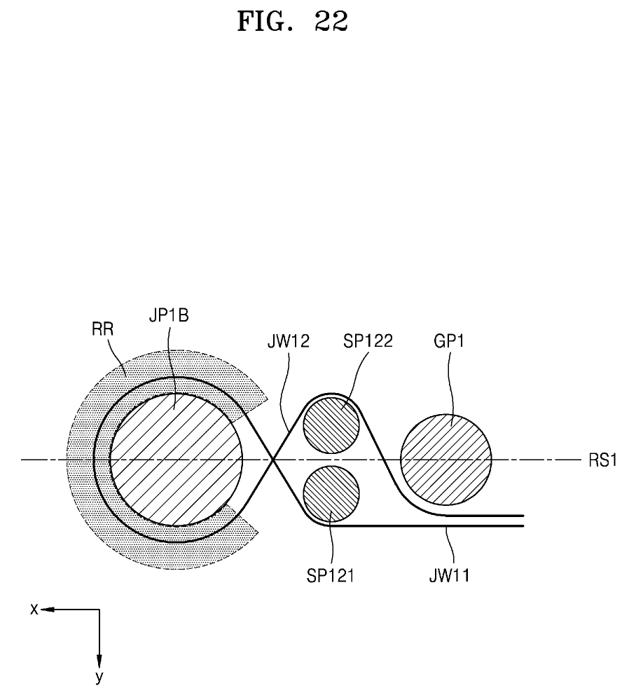

FIGS. 18 and 19 are perspective views illustrating an end tool 120B of a surgical instrument according to another embodiment of the present disclosure, and FIG. 20 is a plan view illustrating the end tool 120B of the surgical instrument of FIG. 18. FIG. 21 is a perspective view illustrating some components of the end tool 120B of the surgical instrument of FIG. 18. FIG. 22 is a conceptual view illustrating a wire guide structure of the end tool 120B of the surgical instrument of FIG. 18.

The end tool 120B of the surgical instrument according to another embodiment of the present disclosure differs mainly in a guide pulley GP disposed in an end tool hub 125B, compared to the end tools 120 and 120A of the above-described embodiments. Accordingly, the following description focuses on these differences, and the descriptions of the other components may be referred to FIGS. 4 and 10 and the like as previously described. That is, the end tool 120B of FIG. 18 includes a first jaw second-1 auxiliary pulley SP121, a first jaw second-2 auxiliary pulley SP122, a second jaw second-1 auxiliary pulley SP221, and a second jaw second-2 auxiliary pulley SP222, as in FIG. 10. However, the present disclosure is not limited thereto, and the following description is equally applicable to the embodiments including the first jaw first auxiliary pulley SP11 and the second jaw first auxiliary pulley SP21, as shown in FIG. 4.

Referring to FIGS. 18 to 22, the end tool 120B according to another embodiment of the present disclosure may include a first jaw auxiliary pulley unit and a second jaw auxiliary pulley unit. The first jaw auxiliary pulley unit and the second jaw auxiliary pulley unit may adjust paths of a first jaw wire JW1 and a second jaw wire JW2, respectively, thereby enabling adjustment of opening and closing radii of a first jaw 121B and a second jaw 122B.

The first jaw wire JW1 extending from a first jaw coupling pulley JP1B may be wound on the first jaw auxiliary pulley unit. The first jaw auxiliary pulley unit may include a plurality of first jaw auxiliary pulleys SP1, and a first jaw first wire portion JW11 and a first jaw second wire portion JW12 of the first jaw wire JW1 may extend while being wound around at least one of the plurality of first jaw auxiliary pulleys SP1. That is, the first jaw auxiliary pulley unit may guide paths of the first jaw first wire portion JW11 and the first jaw second wire portion JW12 extending from the first jaw coupling pulley JP1B.

The second jaw wire JW2 extending from a second jaw coupling pulley JP2B may be wound on the second jaw auxiliary pulley unit. The second jaw auxiliary pulley unit may include a plurality of second jaw auxiliary pulleys SP2, and a second jaw first wire portion JW21 and a second jaw second wire portion JW22 of the second jaw wire JW2 may extend while being wound around at least one of the plurality of second jaw auxiliary pulleys SP2. That is, the second jaw auxiliary pulley unit may guide paths of the second jaw first wire portion JW21 and the second jaw second wire portion JW22 extending from the second jaw coupling pulley JP2B.

The first jaw auxiliary pulley unit may include the first jaw second-1 auxiliary pulley SP121 and the first jaw second-2 auxiliary pulley SP122. The first jaw second-1 auxiliary pulley SP121 and the first jaw second-2 auxiliary pulley SP122 may adjust the paths of the first jaw first wire portion JW11 and the first jaw second wire portion JW12, respectively. The second jaw auxiliary pulley unit may include the second jaw second-1 auxiliary pulley SP221 and the second jaw second-2 auxiliary pulley SP222. The second jaw second-1 auxiliary pulley SP221 and the second jaw second-2 auxiliary pulley SP222 may adjust the paths of the second jaw first wire portion JW21 and the second jaw second wire portion JW22, respectively. Reference may be made to the foregoing description with reference to FIG. 10 and the like for details on the first jaw second-1 auxiliary pulley SP121, the first jaw second-2 auxiliary pulley SP122, the second jaw second-1 auxiliary pulley SP221, and the second jaw second-2 auxiliary pulley SP222.

The first jaw auxiliary pulley unit and the second jaw auxiliary pulley unit may further include a first jaw guide pulley GP1 and a second jaw guide pulley GP2, respectively. The first jaw guide pulley GP1 and the second jaw guide pulley GP2 may be disposed in a pulley coupling portion 12511B of the end tool hub 125B, as shown in FIG. 21, and may each be rotatably coupled to a third auxiliary shaft SS3.

The first jaw guide pulley GP1 may guide the first jaw second wire portion JW12, which extends from another side of a first reference plane RS1 while being wound around the first jaw second-2 auxiliary pulley SP122, to one side of the first reference plane RS1. That is, the first jaw guide pulley GP1 performs substantially the same function as the above-described first main guide groove MG1, and by the first jaw guide pulley GP1, the first jaw first wire portion JW11 and the first jaw second wire portion JW12 may extend from one side of the first reference plane RS1 together to the connection part 140.

The second jaw guide pulley GP2 may guide the second jaw first wire portion JW21, which extends from one side of the first reference plane RS1 while being wound around the second jaw second-1 auxiliary pulley SP221, to another side of the first reference plane RS1. That is, the second jaw guide pulley GP2 performs substantially the same function as the above-described second main guide groove MG2, and by the second jaw guide pulley GP2, the second jaw first wire portion JW21 and the second jaw second wire portion JW22 may extend from another side of the first reference plane RS1 together to the connection part 140.

The rotation of the first jaw 121B and the second jaw 122B according to the arrangement of the paths of the first jaw wire JW1 and the second jaw wire JW2 is summarized as follows.

The first jaw first wire portion JW11 may extend from another side of the first reference plane RS1 while being wound around the first jaw coupling pulley JP1B, and a path thereof may be adjusted by the first jaw second-1 auxiliary pulley SP121 to one side of the first reference plane RS1 and then may extend to the connection part 140. The first jaw second wire portion JW12 may extend from one side of the first reference plane RS1 while being wound around the first jaw coupling pulley JP1B, and a path thereof may be adjusted by the first jaw second-2 auxiliary pulley SP122 to another side of the first reference plane RS1. The first jaw second wire portion JW12 may extend from another side of the first reference plane RS1 while being wound around the first jaw second-1 auxiliary pulley SP121, and a path thereof may be adjusted by the first jaw guide pulley GP1 to one side of the first reference plane RS1 and then may extend to the connection part 140.

Accordingly, the end tool 120B may change both tangential directions of two strands of the first jaw wire JW1 when the two strands extend from the first jaw coupling pulley JP1B. The first jaw wire portions JW11 and JW12 and a coupling member (not shown) may be rotatable until positioned on a common internal tangent of the first jaw coupling pulley JP1B and the first jaw second-1 auxiliary pulley SP121, and may also be rotatable until positioned on a common internal tangent of the first jaw coupling pulley JP1B and the first jaw second-2 auxiliary pulley SP122. Thus, when the first jaw 121B rotates, rotation radii RR in both the clockwise and counterclockwise directions about a jaw rotation shaft 123JA may be increased.

The second jaw first wire portion JW21 may extend from another side of the first reference plane RS1 while being wound around the second jaw coupling pulley JP2B, and a path thereof may be adjusted by the second jaw second-1 auxiliary pulley SP221 to one side of the first reference plane RS1. The second jaw first wire portion JW21 may extend from one side of the first reference plane RS1 while being wound around the second jaw second-1 auxiliary pulley SP221, and a path thereof may be adjusted by the second jaw guide pulley GP2 to another side of the first reference plane RS1 and then may extend toward the connection part 140. The second jaw second wire portion JW22 may extend from one side of the first reference plane RS1 while being wound around the second jaw coupling pulley JP2B, and a path thereof may be adjusted by the second jaw second-1 auxiliary pulley SP221 to another side of the first reference plane RS1 and then may extend to the connection part 140.

Accordingly, when two strands of the second jaw wire JW2 extend from the second jaw coupling pulley JP2B, the end tool 120B is able to change both of their tangential directions. The second jaw wire portions JW21 and JW22 and a coupling member (not shown) may be rotatable until positioned on a common internal tangent of the second jaw coupling pulley JP2B and the second jaw second-1 auxiliary pulley SP221, and may also be rotatable until positioned on a common internal tangent of the second jaw coupling pulley JP2B and the second jaw second-2 auxiliary pulley SP222. Thus, when the second jaw 122B rotates, the rotation radii RR in both the clockwise and counterclockwise directions about the jaw rotation shaft 123JA may be increased.

That is, even when the first jaw 121B and the second jaw 122B are positioned on line M in FIG. 20, the first jaw 121B may be rotatable, and the second jaw 122B may also be additionally rotatable by an angle θ up to line N in FIG. 20. In the opposite situation, the first jaw 121B may also be rotatable, thereby increasing a yaw motion range in which an actuation motion can be performed.

As described with reference to FIGS. 15 to 17, the arrangement of the paths of the first jaw wire JW1 and the second jaw wire JW2 may be variously modified, and an opening and closing radius of the end tool 120B may be adjusted accordingly.

As described above, in the end tool 120B according to an embodiment of the present disclosure, the opening and closing radius may be increased by adjusting the paths of the first jaw wire JW1 and the second jaw wire JW2 through the first jaw second-1 auxiliary pulley SP121, the first jaw second-2 auxiliary pulley SP122, the second jaw second-1 auxiliary pulley SP221, the second jaw second-2 auxiliary pulley SP222, and the first jaw guide pulley GP1 and the second jaw guide pulley GP2 disposed in the end tool hub 125B.

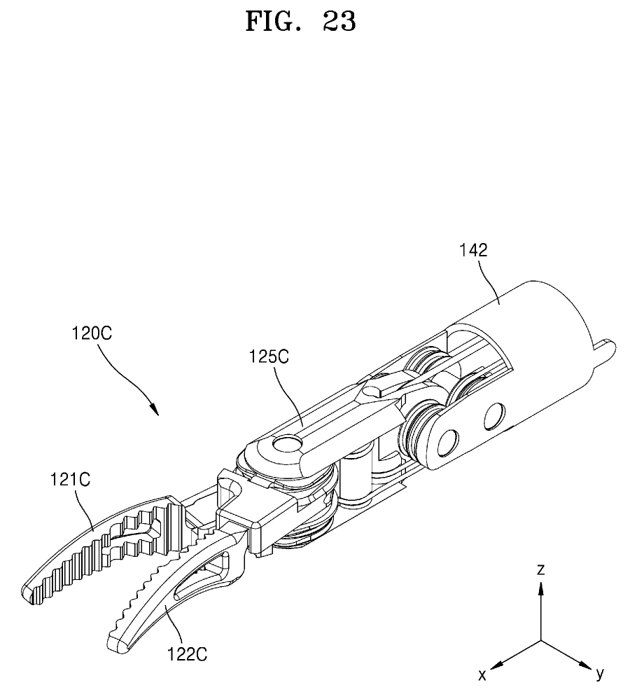

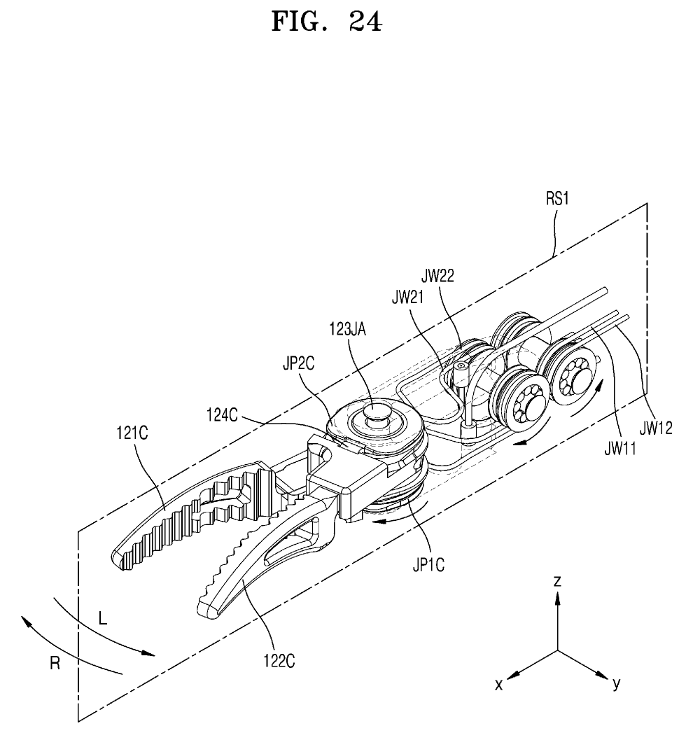

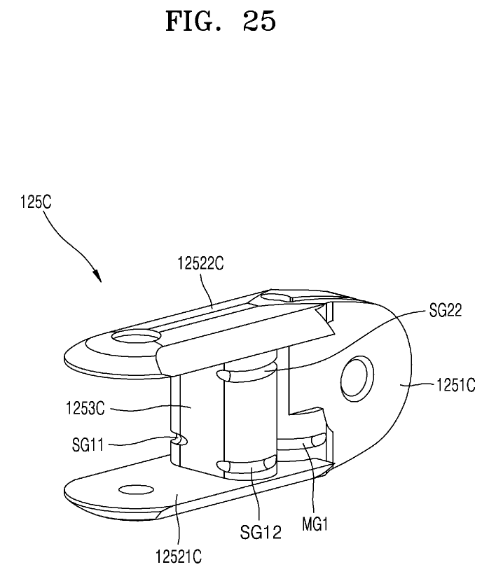

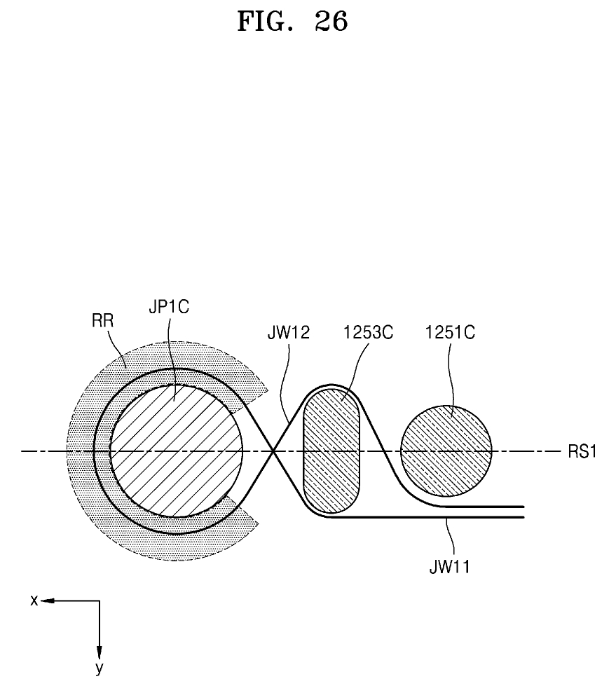

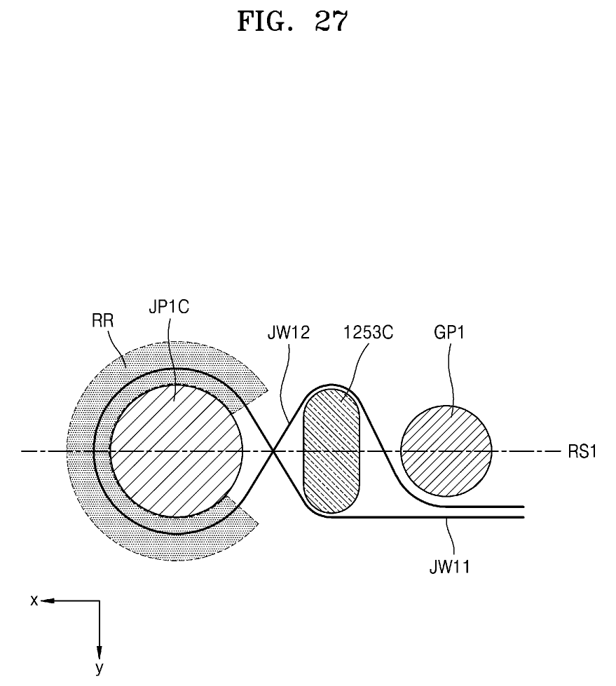

FIGS. 23 and 24 are perspective views illustrating an end tool 120C of a surgical instrument according to another embodiment of the present disclosure, and FIG. 25 is a perspective view illustrating an end tool hub 125C of the end tool 120C of the surgical instrument of FIG. 23. FIG. 26 is a conceptual view illustrating a wire guide structure of the end tool 120C of the surgical instrument of FIG. 23, and FIG. 27 is a view illustrating a modified example of a wire guide structure of the end tool 120C of the surgical instrument of FIG. 23.

The end tool 120C of the surgical instrument according to another embodiment of the present disclosure differs mainly in a structure of guide grooves formed in an end tool hub 125C, compared to the end tools 120A and 120B of the above-described embodiments. Accordingly, the following description focuses on these differences, and the descriptions of the other components may be referred to FIGS. 4, 10, and 18 and the like as previously described.

Referring to FIGS. 23 to 26, the end tool 120C according to another embodiment of the present disclosure may include the end tool hub 125C. The end tool hub 125C serves as a base of the end tool 120C by having a first jaw coupling pulley JP1, a second jaw coupling pulley JP2, and a pitch pulley 123P coupled thereto, and may adjust paths of a first jaw wire JW1 and a second jaw wire JW2.

The end tool hub 125C may include a hub main body 1251C and a pair of hub extensions 12521C and 12522C extending from the hub main body 1251C. The end tool 120C may be shaft-coupled to the connection part hub 142 using the hub main body 1251C, and a first jaw coupling pulley JP1C and a second jaw coupling pulley JP2C may be disposed between the pair of hub extensions 12521C and 12522C and shaft-coupled to the end tool hub 125C.

The end tool hub 125C may further include a hub guide body 1253C. The hub guide body 1253C may be disposed in the form of a pillar connecting the pair of hub extensions 12521C and 12522C to each other. That is, the hub guide body 1253C may be positioned spaced apart from the hub main body 1251C, and the paths of the first jaw wire JW1 and the second jaw wire JW2 may be adjusted between the hub guide body 1253C and the hub main body 1251C.

The hub guide body 1253C may include a plurality of auxiliary guide grooves SG for adjusting the paths of the first jaw wire JW1 and the second jaw wire JW2. The auxiliary guide grooves SG may be formed as recessed grooves in the hub guide body 1253C to guide the paths of the first jaw wire JW1 and the second jaw wire JW2, which respectively extend from the first jaw coupling pulley JP1 and the second jaw coupling pulley JP2. The first jaw wire JW1 and the second jaw wire JW2 may intersect each other in their paths and may be wound around the auxiliary guide grooves SG of the end tool hub 125C. That is, the hub guide body 1253C having the auxiliary guide grooves SG may guide the paths of the first jaw wire JW1 and the second jaw wire JW2 and may perform substantially the same function as the first jaw auxiliary pulley and the second jaw auxiliary pulley described above.

The hub guide body 1253C may include a first-1 auxiliary guide groove SG11, a first-2 auxiliary guide groove SG12, a second-1 auxiliary guide groove SG21, and a second-2 auxiliary guide groove SG22.

The first-1 auxiliary guide groove SG11 may guide a path of a first jaw first wire portion JW11, which extends from another side of a first reference plane RS1 while being wound around the first jaw coupling pulley JP1C, to one side of the first reference plane RS1. The first-2 auxiliary guide groove SG12 may guide a path of a first jaw second wire portion JW12, which extends from one side of the first reference plane RS1 while being wound around the first jaw coupling pulley JP1C, to another side of the first reference plane RS1. In this case, the first-1 auxiliary guide groove SG11 and the first-2 auxiliary guide groove SG12 may be disposed at different heights with respect to a second reference plane (not shown), thereby preventing interference between the path of the first jaw first wire portion JW11 and the path of the first jaw second wire portion JW12. That is, the paths of the first jaw first wire portion JW11 and the first jaw second wire portion JW12, both extending from the first jaw coupling pulley JP1C, may intersect by the first-1 auxiliary guide groove SG11 and the first-2 auxiliary guide groove SG12.

The second-1 auxiliary guide groove SG21 may guide a path of a second jaw first wire portion JW21, which extends from another side of the first reference plane RS1 while being wound around the second jaw coupling pulley JP2C, to one side of the first reference plane RS1. The second-2 auxiliary guide groove SG22 may guide a path of a second jaw second wire portion JW22, which extends from one side of the first reference plane RS1 while being wound around the second jaw coupling pulley JP2C, to another side of the first reference plane RS1. In this case, the second-1 auxiliary guide groove SG21 and the second-2 auxiliary guide groove SG22 may be disposed at different heights with respect to the second reference plane (not shown), thereby preventing interference between the paths of the second jaw first wire portion JW21 and the second jaw second wire portion JW22. That is, the paths of the second jaw first wire portion JW21 and the second jaw second wire portion JW22, both extending from the second jaw coupling pulley JP2C, may intersect by the second-1 auxiliary guide groove SG21 and the second-2 auxiliary guide groove SG22.

The end tool hub 125C may include a first main guide groove MG1 and a second main guide groove MG2.

The first main guide groove MG1 may guide the first jaw second wire portion JW12, which extends from another side of the first reference plane RS1 while being wound around the first-1 auxiliary guide groove SG11, to one side of the first reference plane RS1. That is, by the first main guide groove MG1, the first jaw first wire portion JW11 and the first jaw second wire portion JW12 may extend from one side of the first reference plane together to the connection part 140.

The second main guide groove MG2 may guide the second jaw first wire portion JW21, which extends from one side of the first reference plane RS1 while being wound around the second-2 auxiliary guide groove SG22, to another side of the first reference plane RS1. That is, by the second main guide groove MG2, the second jaw first wire portion JW21 and the second jaw second wire portion JW22 may extend from another side of the first reference plane RS1 together to the connection part 140.

Referring to FIG. 27, in the end tool hub 125C, a first jaw guide pulley GP1 and a second jaw guide pulley GP2 may be disposed in place of the main guide grooves MG.

The first jaw guide pulley GP1 may guide the first jaw second wire portion JW12, which extends from another side of the first reference plane RS1 while being wound around the first-1 auxiliary guide groove SG11, to one side of the first reference plane RS1. That is, by the first jaw guide pulley GP1, the first jaw first wire portion JW11 and the first jaw second wire portion JW12 may extend from one side of the first reference plane together to the connection part 140.

The second jaw guide pulley GP2 may guide the second jaw first wire portion JW21, which extends from one side of the first reference plane RS1 while being wound around the second-2 auxiliary guide groove SG22, to another side of the first reference plane RS1. That is, by the second jaw guide pulley GP2, the second jaw first wire portion JW21 and the second jaw second wire portion JW22 may extend from another side of the first reference plane RS1 together to the connection part 140.

The rotation of a first jaw 121C and a second jaw 122C according to the arrangement of the paths of the first jaw wire JW1 and the second jaw wire JW2 is summarized as follows.

The first jaw first wire portion JW11 may extend from another side of the first reference plane RS1 while being wound around the first jaw coupling pulley JP1C, and a path thereof may be adjusted by the first-2 auxiliary guide groove SG12 to one side of the first reference plane RS1 and then may extend to the connection part 140. The first jaw second wire portion JW12 may extend from one side of the first reference plane RS1 while being wound around the first jaw coupling pulley JP1C, and a path thereof may be adjusted by the first-1 auxiliary guide groove SG11 to another side of the first reference plane RS1. The first jaw second wire portion JW12 may have its path adjusted from another side of the first reference plane RS1 to one side of the first reference plane RS1 by the first main guide groove MG1 or the first jaw guide pulley GP1, and may extend to the connection part 140.

Accordingly, the end tool 120C may change both tangential directions of two strands of the first jaw wire JW1 when the two strands extend from the first jaw coupling pulley JP1C. Thus, when the first jaw 121C rotates, rotation radii RR in both the clockwise and counterclockwise directions about a jaw rotation shaft 123JA may be increased.

The second jaw first wire portion JW21 may extend from another side of the first reference plane RS1 while being wound around the second jaw coupling pulley JP2C, and a path thereof may be adjusted by the second-2 auxiliary guide groove SG22 to one side of the first reference plane RS1. The second jaw first wire portion JW21 may have its path adjusted from one side of the first reference plane RS1 to another side of the first reference plane RS1 by the second main guide groove MG2 or the second jaw guide pulley GP2, and may extend to the connection part 140. The second jaw second wire portion JW22 may extend from one side of the first reference plane RS1 while being wound around the second jaw coupling pulley JP2C, and a path thereof may be adjusted by the second-1 auxiliary guide groove SG21 to another side of the first reference plane RS1 and then may extend toward the connection part 140.

Accordingly, when two strands of the second jaw wire JW2 extend from the second jaw coupling pulley JP2C, the end tool 120C is able to change both of their tangential directions. Thus, when the second jaw 122C rotates, the rotation radii RR in both the clockwise and counterclockwise directions about the jaw rotation shaft 123JA may be increased.

Up to this point, various embodiments have been described regarding the detailed structure of the end tool 120 having a configuration for guiding the paths of the first jaw wire JW1 and the second jaw wire JW2, as well as the wire guiding principle. The end tool 120 of the surgical instrument 100 according to the present disclosure may include a wire guide unit formed by combining auxiliary pulleys, guide pulleys, and guide grooves. The wire guide unit may adjust extension directions of the first jaw wire JW1 and the second jaw wire JW2, which respectively extend from the first jaw coupling pulley JP1 and the second jaw coupling pulley JP2, thereby increasing the rotation radii of the first jaw 121 and the second jaw 122.

Hereinafter, the structure and operational principle of the manipulation part 110, which is connected to the end tool 120 via the jaw wires JW and configured to control the rotational motion of the end tool 120, will be described.

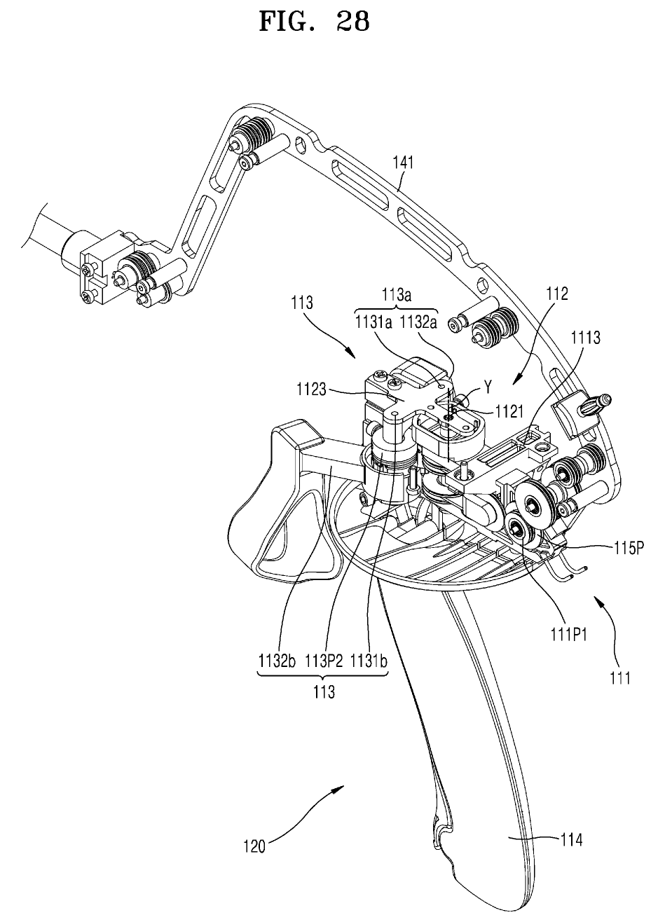

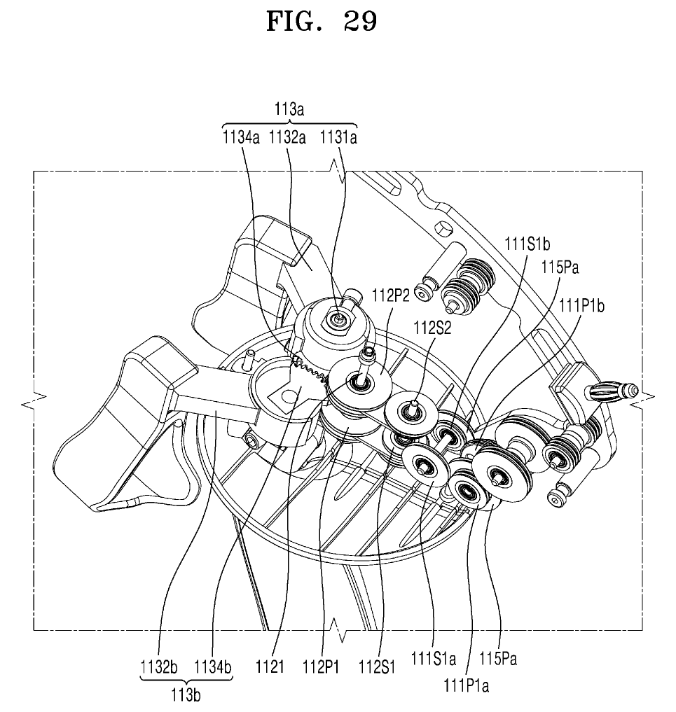

FIGS. 28 and 29 are views illustrating the manipulation part 110 of the surgical instrument 100 of FIG. 2.

Referring to FIGS. 28 and 29, the manipulation part 110 of the surgical instrument 100 according to an embodiment of the present disclosure may include a first handle 114 grippable by a user, the actuation manipulation part 113 configured to control an actuation motion of the end tool 120, the yaw manipulation part 112 configured to control a yaw motion of the end tool 120, and the pitch manipulation part 111 configured to control a pitch motion of the end tool 120.

First, in an example usage of the surgical instrument 100, a user may grip the first handle 114 with the palm, perform a pitch motion by rotating the first handle 114 about the Y-axis, i.e., a pitch rotation axis, and perform a yaw motion by rotating it about the Z-axis, i.e., a yaw rotation axis. For example, the user may insert the thumb and index finger into the actuation manipulation part 113 and perform an actuation motion by rotating the actuation manipulation part 113.

Here, in the surgical instrument 100 according to an embodiment of the present disclosure, when the manipulation part 110 is rotated in one direction with respect to the connection part 140, the end tool 120 is rotated in a direction that is intuitively the same as a manipulation direction of the manipulation part 110. In other words, when the first handle 114 of the manipulation part 110 is rotated in one direction, the end tool 120 is also rotated in a direction intuitively the same as the one direction, so that a pitch motion or a yaw motion is performed. Here, the phrase "intuitively the same direction" may be further explained as meaning that a direction of movement of the user's finger gripping the manipulation part 110 and a direction of movement of a distal end of the end tool 120 form substantially the same direction. Of course, "the same direction" as used herein may not be a perfectly matching direction on a three-dimensional coordinate, and may be understood to be equivalent to the extent that, for example, when the user's finger moves to the left, the distal end of the end tool 120 is moved to the left, and when the user's finger moves down, the end portion of the end tool 120 is moved down.

For example, to this end, in the surgical instrument 100 according to an embodiment of the present disclosure, the manipulation part 110 and the end tool 120 are formed in the same direction with respect to a plane perpendicular to the extension axis (an X-axis) of the connection part 140. That is, when viewed based on a YZ plane of FIG. 2, the manipulation part 110 is configured to extend in a positive (+) X-axis direction, and the end tool 120 is also configured to extend in the positive (+) X-axis direction. In other words, it may be said that a formation direction of the end tool 120 on one end portion of the connection part 140 is the same as a formation direction of the manipulation part 110 on another end portion of the connection part 140 on the basis of the YZ plane. In other words, it may be said that the manipulation part 110 may be formed in a direction away from the body of a user holding the manipulation part 110, that is, in a direction in which the end tool 120 is formed. That is, the first handle 114, actuation rotation parts 1132a and 1132b, or the like, which the user grips and moves to perform the actuation, yaw, and pitch motions, is formed such that the portion that moves to perform each motion extends in the positive (+) X-axis direction beyond the center of rotation of each joint for that motion. In this manner, the manipulation part 110 may be configured in the same manner as the end tool 120 in which each moving portion is configured to extend in the positive (+) X-axis direction from the rotation center of the corresponding joint for the motion, and as described with reference to FIGS. 1A to 1F, the manipulation direction of the user may be identical to the operation direction of the end tool from the viewpoint of the rotation directions and the left and right directions. As a result, intuitively the same manipulation may be achieved.

Actuation, yaw, and pitch motions in the present embodiment will be described as follows.

First, the actuation motion is described as follows.

When the user inserts the index finger into a first actuation rotating part 1132a and the thumb into a second actuation rotating part 1132b, and rotates the actuation rotating parts 1132a and 1132b using one or both of the fingers, a first actuation pulley 113P1 and a first actuation gear 1134a, which are coupled to the first actuation rotating part 1132a, rotate around a first actuation rotation shaft 1131a, and a second actuation pulley 113P2 and a second actuation gear 1134b, which are coupled to the second actuation rotating part 1132b, rotate around a second actuation rotation shaft 1131b.

In this case, the first actuation pulley 113P1 and the second actuation pulley 113P2 rotate in opposite directions, and accordingly, the first jaw wire JW1, which has one end portion coupled to and wound around the first actuation pulley 113P1, and the second jaw wire JW2, which has one end portion coupled to and wound around the second actuation pulley 113P2, also move in opposite directions

For example, a rotating force is transmitted to the end tool 120 through the power transmission part 130, and the two jaws 121 and 122 of the end tool 120 perform the actuation motion. Here, as described above, the actuation motion refers to a motion in which the two jaws 121 and 122 are splayed or closed while being rotated in opposite directions. That is, when the actuation rotating parts 1132a and 1132b of the actuation manipulation part 113 are rotated toward each other, the first jaw 121 rotates in the counterclockwise direction and the second jaw 122 rotates in the clockwise direction, causing the end tool 120 to close. When the actuation rotating parts 1132a and 1132b of the actuation manipulation part 113 are rotated away from each other, the first jaw 121 rotates in the clockwise direction and the second jaw 122 rotates in the counterclockwise direction, causing the end tool 120 to open. In the present embodiment, for the actuation manipulation described above, the first actuation rotating part 1132a and the second actuation rotating part 1132b are provided to configure the second handle and manipulated by gripping the second handle with two fingers. However, for the actuation manipulation in which the two jaws of the end tool 120 are opened or closed, the actuation manipulation part 113 may be configured in a manner different from the above-described manner, such as configuring the two actuation pulleys (the first actuation pulley 113P1 and the second actuation pulley 113P2) to act in opposition to each other with one actuation rotating part.

Next, the yaw motion is described as follows.

When a user rotates the first handle 114 around a yaw rotation shaft 1121 while holding the first handle 114, the actuation manipulation part 113 and the yaw manipulation part 112 are yaw-rotated around the yaw rotation shaft 1121. When the first actuation pulley 113P1 of a first actuation manipulation part 113a, to which the first jaw wire JW1 is coupled, rotates around the yaw rotation shaft 1121, the first jaw wire JW1 wound around a first jaw yaw pulley 112P1 moves accordingly. Similarly, when the second actuation pulley 113P2 of a second actuation manipulation part 113b, to which the second jaw wire JW2 is coupled, rotates around the yaw rotation shaft 1121, the second jaw wire JW2 wound around a second jaw yaw pulley 112P2 moves accordingly. In this case, the first jaw wire JW1 connected to the first jaw 121 and the second jaw wire JW2 connected to the second jaw 122 are wound around the first jaw yaw pulley 112P1 and the second jaw yaw pulley 112P2, respectively, so that the first jaw 121 and the second jaw 122 rotate in the same direction during yaw rotation. For example, a rotating force is transmitted to the end tool 120 through the power transmission part 130, and thus a yaw motion in which the two jaws 121 and 122 of the end tool 120 are rotated in the same direction is performed.

In this case, since a yaw frame 1123 connects the first handle 114, the yaw rotation shaft 1121, the first actuation rotation shaft 1131a, and the second actuation rotation shaft 1131b to each other, the first handle 114, the yaw manipulation part 112, and the actuation manipulation part 113 rotate together around the yaw rotation shaft 1121.

Next, the pitch motion is described as follows.

When a user rotates the first handle 114 around a pitch rotation shaft 1111 while holding the first handle 114, the actuation manipulation part 113, the yaw manipulation part 112, and the pitch manipulation part 111 are pitch-rotated around the pitch rotation shaft 1111. That is, when the first actuation pulley 113P1 of the first actuation manipulation part 113a, to which the first jaw wire JW1 is coupled, rotates around the pitch rotation shaft 1111, the first jaw wire JW1 wound around a first jaw pitch pulley-a 111P1a and a first jaw pitch pulley-b 111P1b moves. Similarly, when the second actuation pulley 113P2 of the second actuation manipulation part 113b, to which the second jaw wire JW2 is coupled, rotates around the pitch rotation shaft 1111, the second jaw wire JW2 wound around a second jaw pitch pulley-a 111P2a and a second jaw pitch pulley-b 111P2b moves. In this case, as described with reference to FIG. 9, two strands of the first jaw wire JW1 move in the same direction, and two strands of the second jaw wire JW2 also move in the same direction. Accordingly, the first jaw wire JW1 and the second jaw wire JW2 are wound around the first jaw pitch pulleys 111P1a and 111P1b and the second jaw pitch pulleys 111P2a and 111P2b, respectively, so that the first jaw 121 and the second jaw 122 perform a pitch rotation. For example, a rotating force is transmitted to the end tool 120 through the power transmission part 130, and the two jaws 121 and 122 of the end tool 120 perform the pitch motion.

At this time, since a pitch frame 1113 is connected to the yaw frame 1123, and the yaw frame 1123 connects the first handle 114, the yaw rotation shaft 1121, the first actuation rotation shaft 1131a, and the second actuation rotation shaft 1131b to each other, when the pitch frame 1113 rotates around the pitch rotation shaft 1111, the pitch frame 1113, the first handle 114, the yaw rotation shaft 1121, the first actuation rotation shaft 1131a, and the second actuation rotation shaft 1131b connected to the yaw frame 1123 all rotate together. That is, when the pitch manipulation part 111 rotates around a pitch rotation shaft 1111, the actuation manipulation part 113 and the yaw manipulation part 112 rotate together with the pitch manipulation part 111.

In summary, in the surgical instrument 100 according to an embodiment of the present disclosure, the pulleys are formed on respective joint points (an actuation joint, a yaw joint, and a pitch joint), the wires (the first jaw wire or the second jaw wire) are wound around the pulleys, the rotational manipulations (actuation rotation, yaw rotation, and pitch rotation) of the manipulation part cause the movement of each wire, which in turn induces the desired motion of the end tool 120. Furthermore, the auxiliary pulley may be formed at one side of each of the pulleys, and the wire may not be wound several times around one pulley due to the auxiliary pulley.

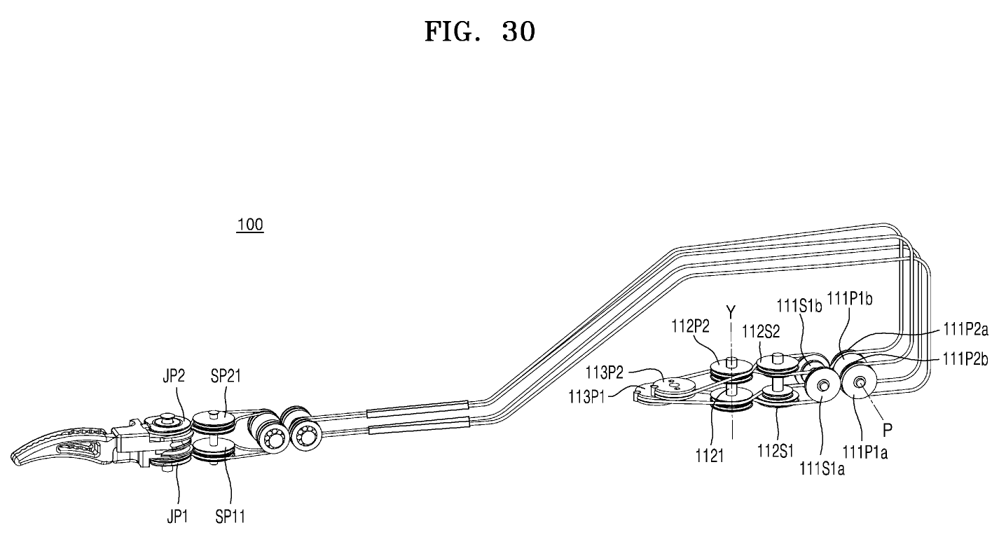

FIG. 30 is a view schematically illustrating only a configuration of the pulleys and wires constituting joints of the surgical instrument 100 according to an embodiment of the present disclosure illustrated in FIG. 28.

Referring to FIG. 30, the manipulation part 110 may include the first actuation pulley 113P1, the first jaw yaw pulley 112P1, a first jaw yaw auxiliary pulley 112S1, the first jaw pitch pulley-a 111P1a, a first jaw pitch pulley-b 111P1b, a first jaw pitch auxiliary pulley-a 111S1a, and a first jaw pitch auxiliary pulley-b 111S1b, all of which are associated with a rotational motion of the first jaw 121.

The manipulation part 110 may also include the second actuation pulley 113P2, the second jaw yaw pulley 112P2, a second jaw yaw auxiliary pulley 112S2, the second jaw pitch pulley-a 111P2a, a second jaw pitch pulley-b 111P2b, a second jaw pitch auxiliary pulley-a 111S2a, and a second jaw pitch auxiliary pulley-b 111S2b, all of which are associated with the rotational motion of the second jaw 122. (the arrangement and structure of each of the pulleys of the manipulation part 110 are the same in principle as the arrangement and structure of each of the pulleys of the end tool 120, and thus specific designations of some reference numerals are omitted in the drawings).

The first jaw yaw pulley 112P1 and the second jaw yaw pulley 112P2 may be configured to be rotatable independently of each other around the same shaft, which is the yaw rotation shaft 1121. In this case, the first jaw yaw pulley 112P1 and the second jaw yaw pulley 112P2 may be formed as two pulleys that face each other and are capable of rotating independently.

The first jaw yaw auxiliary pulley 112S1 and the second jaw yaw auxiliary pulley 112S2 may be configured to be rotatable independently of each other around the same shaft. Here, the first jaw yaw auxiliary pulley 112S1 may be formed as two pulleys configured to face each other and configured to be independently rotatable, and in this case, the two pulleys may be configured to have different diameters. Similarly, the second jaw yaw auxiliary pulley 112S2 may be formed as two pulleys configured to face each other and configured to be independently rotatable, and in this case, the two pulleys may be configured to have different diameters.

The first jaw pitch auxiliary pulley-a 111S1a, the first jaw pitch auxiliary pulley-b 111S1b, the second jaw pitch auxiliary pulley-a 111S2a, and the second jaw pitch auxiliary pulley-b 111S2b may be configured to be rotatable independently of each other around the same shaft. In this case, the first jaw pitch auxiliary pulley-a 111S1a and the first jaw pitch auxiliary pulley-b 111S1b may have different diameters. The second jaw pitch auxiliary pulley-a 111S2a and the second jaw pitch auxiliary pulley-b 111S2b may also have different diameters.

The first jaw pitch pulley-a 111P1a, the first jaw pitch pulley-b 111P1b, the second jaw pitch pulley-a 111P2a, and the second jaw pitch pulley-b 111P2b may be configured to be rotatable independently of each other around the same shaft, which is the pitch rotation shaft 1111.

The first jaw wire JW1 passes through the first jaw pitch pulley-a 111P1a, the first jaw pitch auxiliary pulley-a 111S1a, the first jaw yaw auxiliary pulley 112S1, and the first jaw yaw pulley 112P1 of the manipulation part 110 in sequence, and is then wound around the first actuation pulley 113P1. After that, the first jaw wire JW1 passes again through the first jaw yaw pulley 112P1, the first jaw yaw auxiliary pulley 112S1, the first jaw pitch auxiliary pulley-b 111S1b, and the first jaw pitch pulley-b 111P1b in sequence, thereby allowing the first jaw wire JW1 to move along the above pulleys while rotating the above pulleys. In this case, the first jaw wire JW1 may be coupled to a specific point on the first actuation pulley 113P1.

The second jaw wire JW2 passes through the second jaw pitch pulley-a 111P2a, the second jaw pitch auxiliary pulley-a 111S2a, the second jaw yaw auxiliary pulley 112S2, and the second jaw yaw pulley 112P2 of the manipulation part 110 in sequence, and is then wound around the second actuation pulley 113P2. After that, the second jaw wire JW2 passes again through the second jaw yaw pulley 112P2, the second jaw yaw auxiliary pulley 112S2, the second jaw pitch auxiliary pulley-b 111S2b, and the second jaw pitch pulley-b 111P2b in sequence, thereby allowing the second jaw wire JW2 to move along the above pulleys while rotating the above pulleys. In this case, the second jaw wire JW2 may be coupled to a specific point on the second actuation pulley 113P2.

As described above, the surgical instrument 100 is capable of independently performing yaw, pitch, and actuation motions through the pulley and wire structure described above, and the user can intuitively manipulate the surgical instrument 100 through corresponding movements.

The end tool of the surgical instrument according to an embodiment of the present disclosure includes a pair of jaws that are rotatable and is configured to increase rotation radii of the jaws by adjusting the arrangement of jaw wire paths. The end tool of the surgical instrument according to an embodiment of the present disclosure may include a wire guide unit including auxiliary pulleys and/or guide grooves formed in an end tool hub. Since the rotation radii of the jaws may vary depending on extension directions of the jaw wires wound around and extending from jaw coupling pulleys, the end tool may adjust the extension directions of the jaw wires through the wire guide unit to adjust an opening and closing radius of the end tool.

In an end tool of a surgical instrument according to an embodiment of the present disclosure, opening and closing radii of a pair of jaws can be increased by adjusting the arrangement of jaw wire paths. In an end tool of a surgical instrument according to an embodiment of the present disclosure, rotation radii of jaws can be adjusted by adjusting extension directions of jaw wires through auxiliary pulleys and/or guide grooves formed in an end tool hub.

In an end tool of a surgical instrument according to an embodiment of the present disclosure, a yaw motion range in which an actuation motion can be performed can be increased by adjusting the arrangement of jaw wire paths. In an end tool of a surgical instrument according to an embodiment of the present disclosure, jaw wire paths can be arranged so as not to interfere with each other, thereby allowing power to be safely and efficiently transmitted.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. An end tool for a surgical instrument, the end tool comprising:
a first jaw and a second jaw, both configured to be rotatable independently of each other;
a first jaw coupling pulley coupled to the first jaw;
a first jaw wire coupled to the first jaw coupling pulley, and having a first jaw first wire portion and a first jaw second wire portion;
a second jaw coupling pulley coupled to the second jaw;
a second jaw wire coupled to the second jaw coupling pulley, and having a second jaw first wire portion and a second jaw second wire portion;
a first jaw auxiliary pulley assembly around which the first jaw wire extending from the first jaw coupling pulley is wound, and configured to adjust a first path of the first jaw first wire portion and a second path of the first jaw second wire portion;
a second jaw auxiliary pulley assembly around which the second jaw wire extending from the second jaw coupling pulley is wound, and configured to adjust a third path of the second jaw first wire portion and a fourth path of the second jaw second wire portion; and
an end tool hub having the first jaw coupling pulley, the second jaw coupling pulley, the first jaw auxiliary pulley assembly, and the second jaw auxiliary pulley assembly rotatably disposed thereon, the end tool hub configured to:
guide the first path of the first jaw first wire portion and the second path of the first jaw second wire portion, both extending from the first jaw auxiliary pulley assembly, to one side of a first reference plane, wherein the first reference plane is defined as one imaginary plane, and
guide the third path of the second jaw first wire portion and the fourth path of the second jaw second wire portion, both extending from the second jaw auxiliary pulley assembly, to another side of the first reference plane.

2. The end tool of claim 1, wherein
the first jaw auxiliary pulley assembly includes a first jaw first auxiliary pulley configured to:
guide the first jaw first wire portion, which extends from the another side of the first reference plane while being wound around the first jaw coupling pulley, to the one side of the first reference plane, and
guide the first jaw second wire portion, which extends from the one side of the first reference plane while being wound around the first jaw coupling pulley, to the another side of the first reference plane, and
the second jaw auxiliary pulley assembly includes a second jaw first auxiliary pulley configured to:
guide the second jaw first wire portion, which extends from the another side of the first reference plane while being wound around the second jaw coupling pulley, to the one side of the first reference plane, and
guide the second jaw second wire portion, which extends from the one side of the first reference plane while being wound around the second jaw coupling pulley, to the another side of the first reference plane.

3. The end tool of claim 2, wherein the end tool hub includes:
a first main guide groove defined on the one side of the first reference plane and configured to guide the second path of the first jaw second wire portion extending from the first jaw first auxiliary pulley; and
a second main guide groove defined on the another side of the first reference plane and configured to guide the third path of the second jaw first wire portion extending from the second jaw first auxiliary pulley.

4. The end tool of claim 2, wherein
the first jaw auxiliary pulley assembly further includes a first jaw guide pulley rotatably coupled to the end tool hub, the first jaw guide pulley configured to guide the second path of the first jaw second wire portion, which extends from the first jaw first auxiliary pulley, to the one side of the first reference plane, and
the second jaw auxiliary pulley assembly further includes a second jaw guide pulley rotatably coupled to the end tool hub, the second jaw guide pulley configured to guide the third path of the second jaw first wire portion, which extends from the second jaw first auxiliary pulley, to the one side of the first reference plane.

5. The end tool of claim 1, wherein
the first jaw auxiliary pulley assembly includes:
one first jaw second auxiliary pulley disposed on the one side of the first reference plane and around which the first jaw first wire portion, which extends while being wound around the first jaw coupling pulley, is wound; and
another first jaw second auxiliary pulley disposed on the another side of the first reference plane and around which the first jaw second wire portion, which extends while being wound around the first jaw coupling pulley, is wound, and
the second jaw auxiliary pulley assembly includes:
one second jaw second auxiliary pulley disposed on the one side of the first reference plane and around which the second jaw first wire portion, which extends while being wound around the second jaw coupling pulley, is wound; and
another second jaw second auxiliary pulley disposed on the other side of the first reference plane and around which the second jaw second wire portion, which extends while being wound around the second jaw coupling pulley, is wound.

6. The end tool of claim 5, wherein
the one first jaw second auxiliary pulley and the one second jaw second auxiliary pulley are rotatably coupled to a first auxiliary shaft disposed in the end tool hub, and
the another first jaw second auxiliary pulley and the another second jaw second auxiliary pulley are rotatably coupled to a second auxiliary shaft disposed in the end tool hub.

7. The end tool of claim 6, wherein
the one first jaw second auxiliary pulley and the another first jaw second auxiliary pulley are disposed at different heights from a second reference plane, wherein the second reference plane is defined as another imaginary plane to prevent interference with each other, and
the one second jaw second auxiliary pulley and the another second jaw second auxiliary pulley are disposed at different heights from the second reference plane to prevent interference with each other.

8. The end tool of claim 6, wherein
the one first jaw second auxiliary pulley and the another first jaw second auxiliary pulley are disposed in a tilted arrangement to prevent interference with each other, and
the one second jaw second auxiliary pulley and the another second jaw second auxiliary pulley are disposed in the tilted arrangement to prevent interference with each other.

9. The end tool of claim 5, wherein the end tool hub includes:
a first main guide groove defined on the one side of the first reference plane and configured to guide the second path of the first jaw second wire portion extending from the another first jaw second auxiliary pulley; and
a second main guide groove defined on the another side of the first reference plane and configured to guide the third path of the second jaw first wire portion extending from the one second jaw second auxiliary pulley.

10. The end tool of claim 5, wherein
the first jaw auxiliary pulley assembly further includes a first jaw guide pulley rotatably coupled to the end tool hub and configured to guide the second path of the first jaw second wire portion, which extends from the another first jaw second auxiliary pulley, to the one side of the first reference plane, and
the second jaw auxiliary pulley assembly further includes a second jaw guide pulley rotatably coupled to the end tool hub and configured to guide the third path of the second jaw first wire portion, which extends from the one second jaw second auxiliary pulley, to the another side of the first reference plane.

11. The end tool of claim 1, wherein
the first jaw auxiliary pulley assembly includes a first jaw third auxiliary pulley configured to:
guide the first jaw first wire portion, which extends from the another side of the first reference plane while being wound around the first jaw coupling pulley, to the one side of the first reference plane, and
guide the first jaw second wire portion, which extends from the one side of the first reference plane while being wound around the first jaw coupling pulley, to the one side of the first reference plane, and
the second jaw auxiliary pulley assembly includes a second jaw third auxiliary pulley configured to:
guide the second jaw first wire portion, which extends from the another side of the first reference plane while being wound around the second jaw coupling pulley, to the another side of the first reference plane, and
guide the second jaw second wire portion, which extends from the one side of the first reference plane while being wound around the second jaw coupling pulley, to the another side of the first reference plane.

12. An end tool for a surgical instrument, the end tool comprising:
a first jaw and a second jaw, both configured to be rotatable independently of each other;
a first jaw coupling pulley coupled to the first jaw;
a first jaw wire coupled to the first jaw coupling pulley, and having a first jaw first wire portion and a first jaw second wire portion;
a second jaw coupling pulley coupled to the second jaw;
a second jaw wire coupled to the second jaw coupling pulley, and having a second jaw first wire portion and a second jaw second wire portion; and
an end tool hub having the first jaw coupling pulley and the second jaw coupling pulley rotatably disposed thereon, the end tool hub configured to:
guide the first jaw wire to one side of a first reference plane, wherein the first reference plane is defined as one imaginary plane,
guide the second jaw wire to another side of the first reference plane,
adjust a first path of the first jaw first wire portion and a second path of the first jaw second wire portion to intersect each other, and
adjust a third path of the second jaw first wire portion and a fourth path of the second jaw second wire portion to intersect each other.

13. The end tool of claim 12, wherein
the end tool hub includes:
a hub main body; and
a hub guide body extending from the hub main body and interconnecting a pair of hub extensions that face each other,
wherein the hub guide body includes:
one first auxiliary guide groove configured to guide the first path of the first jaw first wire portion, which extends from the another side of the first reference plane while being wound around the first jaw coupling pulley, to the one side of the first reference plane;
another first auxiliary guide groove configured to guide the second path of the first jaw second wire portion, which extends from the one side of the first reference plane while being wound around the first jaw coupling pulley, to the another side of the first reference plane;
one second auxiliary guide groove configured to guide the third path of the second jaw first wire portion, which extends from the another side of the first reference plane while being wound around the second jaw coupling pulley, to the one side of the first reference plane; and
another second auxiliary guide groove configured to guide the fourth path of the second jaw second wire portion, which extends from the one side of the first reference plane while being wound around the second jaw coupling pulley, to the another side of the first reference plane.

14. The end tool of claim 13, wherein
the one first auxiliary guide groove and the another first auxiliary guide groove are disposed at different heights from a second reference plane, wherein the second reference plane is defined as another imaginary plane, and
the one second auxiliary guide groove and the another second auxiliary guide groove are disposed at different heights from the second reference plane.

15. The end tool of claim 13, wherein the hub main body includes:
a first main guide groove defined on the one side of the first reference plane and configured to guide the second path of the first jaw second wire portion extending from the another first auxiliary guide groove; and
a second main guide groove defined on the another side of the first reference plane and configured to guide the third path of the second jaw first wire portion extending from the another second auxiliary guide groove.

16. The end tool of claim 13, further comprising:
a first jaw guide pulley rotatably coupled to the hub main body and configured to guide the second path of the first jaw second wire portion, which extends from the another first auxiliary guide groove, to the one side of the first reference plane; and
a second jaw guide pulley rotatably coupled to the hub main body and configured to guide the third path of the second jaw first wire portion, which extends from the one second auxiliary guide groove, to the another side of the first reference plane.

17. An end tool for a surgical instrument, the end tool comprising:
a first jaw and a second jaw, both configured to be rotatable independently of each other;
a first jaw coupling pulley coupled to the first jaw;
a first jaw wire coupled to the first jaw coupling pulley, and having a first jaw first wire portion and a first jaw second wire portion;
a second jaw coupling pulley coupled to the second jaw;
a second jaw wire coupled to the second jaw coupling pulley, and having a second jaw first wire portion and a second jaw second wire portion;
an end tool hub having the first jaw coupling pulley and the second jaw coupling pulley rotatably disposed thereon; and
a wire guide assembly configured to:
adjust a first path of the first jaw first wire portion and a second path of the first jaw second wire portion within the end tool hub, guiding the first jaw wire to extend to an outside of the end tool hub from one side of a first reference plane, wherein the first plane is defined as one imaginary plane, and
adjust a third path of the second jaw first wire portion and a fourth path of the second jaw second wire portion within the end tool hub, guiding the second jaw wire to extend to the outside of the end tool hub from another side of the first reference plane.

18. The end tool of claim 17, wherein the wire guide assembly includes:
a first jaw guide portion configured to adjust the first path of the first jaw first wire portion and the second path of the first jaw second wire portion within the end tool hub to intersect each other; and
a second jaw guide portion configured to adjust the third path of the second jaw first wire portion and the fourth path of the second jaw second wire portion within the end tool hub to intersect each other.
